SEQUENCE LISTING (2 sequences)

SEQ ID NO: 1 - protein, 20 aa, Clostridium difficile
AGAGTTTGATYMTGGCTCAG

SEQ ID NO: 2 - protein, 21 aa, Clostridium difficile
ACGGYTACCTTGTTACGACTT

(19) United States
(12) Patent Application Publication
(10) Pub. No.: US 2026/0144796 A1
(43) Pub. Date: May 28, 2026

(54) Compounds and Methods for Improving Health

(71) Applicant: Acurx Pharmaceuticals, Inc., Staten Island, NY (US)

(72) Inventors: Kevin GAREY, Houston, TX (US); Eugénie Anne Laure BASSÈRES, Houston, TX (US)

(21) Appl. No.: 19/121,573

(22) PCT Filed: Oct. 17, 2023
(86) PCT No.: PCT/US2023/077091

§ 371 Date: Apr. 16, 2025

Related U.S. Application Data
(60) Provisional application No. 63/416,840, filed on Oct. 17, 2022.
(60) Provisional application No. 63/495,243, filed on Apr. 10, 2023.
(60) Provisional application No. 63/538,981, filed on Sep. 18, 2023.

(51) Int. Cl. A61K 31/5377; A61K 31/522; A61P 1/00; A61P 31/04

(52) U.S. Cl. CPC A61K 31/5377; A61K 31/522; A61P 1/00; A61P 31/04

(57) ABSTRACT

The technical field relates to methods for promoting gut microbiome health using low G+C Directed (LDN) analogs. More particularly, the technical field relates to methods of using LDN analogs as inhibitors of DNA Polymerase IIIC (DNA pol IIIC) enzyme to selectively reduce physiologically harmful Gram-positive bacteria with a genome having low amounts of Guanine and Cytosine (rather the genomes comprise a greater amount of adenine and thymine/uracil nucleotides) in the gut microbiome while simultaneously maintaining or increasing the proportions of beneficial microflora.

All publications, patents and patent applications referred to herein are incorporated by reference in their entirety to the same extent as if each individual publication, patent or patent application was specifically and individually indicated to be incorporated by reference in its entirety.

### TECHNICAL FIELD

The technical field relates to methods for promoting gut microbiome health using low G+C Directed (LDN) analogs. More particularly, the technical field relates to methods of using LDN analogs as inhibitors of DNA Polymerase IIIC (DNA pol IIIC) enzyme to selectively reduce physiologically harmful Gram-positive bacteria with a genome having low amounts of Guanine and Cytosine (rather the genomes comprise a greater amount of adenine and thymine/uracil nucleotides) in the gut microbiome while simultaneously maintaining or increasing the proportions of beneficial microflora.

### BACKGROUND

The mucosal surfaces of the body contain complex and specialized microbial communities, often referred to as the microbiome or microbiota (Mullish B H, Quraishi M N, Segal J P, et al., The gut microbiome: what every gastroenterologist needs to know, Frontline Gastroenterology 2021; 12:118-127). The microbiome of the human gastrointestinal tract is estimated to consist of up to 100 trillion microorganisms, most of them found in the large intestine. (Kachrimanidou M, Tsintarakis E. Insights into the Role of Human Gut Microbiota in Clostridioides difficile Infection. Microorganisms. 2020; 8(2):200 https://doi.org/10.3390/microorganisms8020200). While the gastrointestinal microbiome is diverse, in healthy adults it is predominantly composed of bacteria from two major phyla, Firmicutes (Gram-positive spore forming organisms) and Bacteroidetes (Gram-negative non-spore forming organisms). These two phyla typically comprise approximately 90% of the microbiome (Mullish B H, Quraishi M N, Segal J P, et al., The gut microbiome: what every gastroenterologist needs to know, Frontline Gastroenterology 2021; 12:118-127).

In addition to Firmicutes and Bacteroidetes, the gut microbiome also is made up of additional bacteria including Actinobacteria, Fusobacteria, Verrucomicrobia, and Proteobacteria (Mullish B H, Quraishi M N, Segal J P, et al., The gut microbiome: what every gastroenterologist needs to know, Frontline Gastroenterology 2021; 12:118-127). The Proteobacteria phylum is made up of Gram-negative facultative anaerobes. While some members of the Proteobacteria phylum are, at low amounts, part of a healthy gut, this phylum also comprises the common undesirable Gram-negative pathobionts such as Salmonella, Shigella, and Escherichia coli (Mullish B H, Quraishi M N, Segal J P, et al., The gut microbiome: what every gastroenterologist needs to know, Frontline Gastroenterology 2021; 12:118-127).

Actinobacteria are present in large proportion in children and generally decrease in overall proportion with age (replaced by Firmicutes and Bacteroidetes). At birth, facultative anaerobic species such as E. coli, Staphylococcus, and Streptococcus colonize the infant gut and produce anaerobic environs in the first few days of life that allow strict anaerobes (anaerobes that cannot grow in the presence of greater than 5 μM dissolved oxygen) like Bacteroides (Bacteroidetes phylum) and Bifidobacterium (Actinobacteria phylum) to thrive (Mueller N T, et al., The infant microbiome development: mom matters. Trends Mol Med. 2015 February; 21(2):109-17. doi: 10.1016/j.molmed.2014.12.002. Epub 2014 Dec. 11. PMID: 25578246; PMCID: PMC4464665). Over the first year of life, and through the exposure of the infants to the environment and either breast milk or formula, the gut microbiome evolves into the mature biome that approximates the adult gut microbiome. (Jangi, S. and Lamont, T., (2010) Asymptomatic Colonization by Clostridium difficile in Infants: Implications for Disease in Later Life, Journal of Pediatric Gastroenterology and Nutrition. 51 (1): 2-7).

The gut microbiome exhibits a symbiotic relationship with the host. Through this mutualistic relationship, the microbiome provides a number of benefits to the host, including shaping the intestinal and systemic immune system, maintaining the healthy intestinal epithelium, harvesting energy from food and protection against pathogens. (Mullish B H, Quraishi M N, Segal J P, et al., The gut microbiome: what every gastroenterologist needs to know, Frontline Gastroenterology 2021; 12:118-127). When the composition of the microbiome is altered from its normal diversity, these beneficial physiological functions are disrupted. This is called dysbiosis. (Mullish B H, Quraishi M N, Segal J P, et al., The gut microbiome: what every gastroenterologist needs to know, Frontline Gastroenterology 2021; 12:118-127). When the gut microbiome is in a state of dysbiosis, the microbiome has fewer beneficial microbes (symbionts) and more of the potentially harmful microbes (pathobionts). (Mullish B H, Quraishi M N, Segal J P, et al., The gut microbiome: what every gastroenterologist needs to know, Frontline Gastroenterology 2021; 12:118-127).

Important to health are anaerobic bacteria, i.e., those which grow in oxygen-depleted atmospheres, such as those found in intestinal milieu. Gram-positive anaerobes, such as Lactobacilli, Bifidobacteria, and Eubacteria, as well as Gram-negative anaerobes, such as Bacteroides, represent “good” intestinal organisms important to health. In contrast, the Gram-positive anaerobes Clostridioides difficile and Clostridioides perfringens represent pathogenic bacteria. In particular, Clostridioides difficile (C. diff.) has been increasingly associated with disease in patients, likely due to the reliance on antibacterial drugs used to treat pathogenic bacterial infections.

Modern medicine has been shaped by the advent and use of antibiotic drugs. The mid-20th century was even named the “antibiotic era.” In fact, infectious diseases were believed to be eradicated by the end of the last century (Huemer, M. et al., Antibiotic resistance and persistence-Implications for human health and treatment perspectives, EMBO Rep. 2020 Dec. 3; 21(12):e51034). Although antibiotics have been essential in treating and curing an array of bacterial infections, increasing numbers of bacteria are becoming resistant to a growing number of antibiotics currently in use. Such extensive use of antibacterial drugs has resulted in bacterial microorganisms exhibiting multidrug-resistance (MDR) (Tanwar J, Das S, Fatima Z, Hameed S (2014) Multidrug resistance: an emerging crisis. Interdiscip Perspect Infect Dis 2014:541340). Since new antimicrobial drugs are scarce and due to the increasing prevalence of MDR bacteria causing treatment failures, antibiotic-resistant bacteria have become a major threat to modern health care (Spellberg B, Bartlett J G, Gilbert D N (2013) The future of antibiotics and resistance. N Engl J Med 368:299-302). As a result, bacterial pathogens pose a serious threat to public health.

Nosocomial pathogens and infections are those that are hospital-acquired and pose considerable threat to hospital staff and patients. Nosocomial pathogenic bacteria with growing levels of multidrug resistance and virulence are referred to as ESKAPE pathogens (Huemer, M. et al., Antibiotic resistance and persistence-Implications for human health and treatment perspectives, EMBO Rep. 2020 Dec. 3; 21(12): e51034). ESKAPE stands for the Gram-positive bacterial species Enterococcus faecium and S. aureus and the Gram-negative bacteria K. pneumoniae, A. baumannii, P. aeruginosa and Enterobacter species. ESKAPE pathogens place a significant burden on patient health as well as healthcare systems. These pathogens are characterized by high levels of MDR and are responsible for causing hospital-acquired and potentially life-threatening infections in critically ill and immunocompromised patients (Huemer, M. et al., Antibiotic resistance and persistence-Implications for human health and treatment perspectives, EMBO Rep. 2020 Dec. 3; 21 (12): e51034; and Rice L B (2010) Progress and challenges in implementing the research on ESKAPE pathogens. Infect Control Hosp Epidemiol 31(Suppl 1): S7-10). As microbial resistance to many antimicrobial drugs increases, the use of “last-resort” antibiotics, for example, tigecycline, polymyxin E, daptomycin, vancomycin and linezolid, have become more prevalent (Li, W., et al., (2022), Evaluation of culturable ‘last-resort’ antibiotic resistant pathogens in hospital wastewater and implications on the risks of nosocomial antimicrobial resistance prevalence. Journal of hazardous materials, 438, 129477). These so-called last resort antibiotics serve as the “last line of defense” for antibiotic resistant pathogen infections. As a result, the increasing prevalence of ‘last-resort’ antibiotic resistant pathogens in hospital environments and the nosocomial transmission of these pathogens poses a grave threat to the well-being of patients (Li, W., et al., (2022), Evaluation of culturable ‘last-resort’ antibiotic resistant pathogens in hospital wastewater and implications on the risks of nosocomial antimicrobial resistance prevalence. Journal of hazardous materials, 438, 129477).

Two Gram-positive pathogens, Staphylococcus aureus and Enterococcus fecalis/fecium, are primarily nosocomial pathogens; and together, they presently account for the majority of nosocomial diseases. Further, Clostridioides difficile infection (CDI) is the most common cause of healthcare-associated infections in the United States. (Magill, et al., “Changes in Prevalence of Health Care-Associated Infections in U.S. Hospitals” The New England Journal of Medicine 2018, 379, 1732-1744). Importantly, C. difficile may sometimes be a normal component of the healthy gut microbiome. During periods of microbiome dysbiosis (which may be the result of antibiotic treatment), however, C. difficile can thrive and cause disease, CDI.

Overgrowth of C. difficile in the intestinal milieu may result in a wide spectrum of clinical symptoms ranging from mild diarrhea to severe life-threatening colonic perforation and toxic megacolon (extreme inflammation and distention of the colon). Use of last resort antibiotics disrupts the host microbiome such that CDI is free to thrive without competition (Davis M L, et al. Multicenter derivation and validation of a simple predictive index for healthcare-associated C. difficile infection. (Clin Microbiol Infect 2018; 24:1190-4)). Through treatment with broad spectrum antibiotics, for example, there can be an almost total loss of Bacteroidetes, a reduction in Firmicutes and an overgrowth of Proteobacteria; these changes allow the C. difficile spores to germinate, resulting in increased growth and an increased likelihood of pathogenicity (Mullish B H, Quraishi M N, Segal J P, et al., The gut microbiome: what every gastroenterologist needs to know, Frontline Gastroenterology 2021; 12:118-127). Despite the dangers of antimicrobial resistance, and due to limited treatment options, antimicrobial therapy remains the first line of defense against pathogenic microbial infections. For example, antibiotic treatment is still the primary method of treatment for pathogens such as S. aureus, C. difficile, E. fecalis/fecium, and other known bacterial pathogens.

In contrast to Gram-negative bacteria, which are less permeable to many antibacterial compounds (e.g. vancomycin), the Gram-positive pathobiont S. aureus is naturally susceptible to almost every antibiotic that has been developed. Despite the antimicrobial susceptibility, however, it is well known that S. aureus and similar pathobionts quickly acquire antibiotic resistance. Often, such pathobionts acquire antimicrobial resistance by means of obtaining specific genetic modifications, for example attaining advantageous mutations or undergoing horizontal gene transfer. As a result of the back and forth relationship between antimicrobial development and pathobiont evolution, these types of pathogenic bacterial infections tend to occur in epidemic waves (Chambers H F, Deleo F R (2009) Waves of resistance: Staphylococcus aureus in the antibiotic era. Nat Rev Microbiol 7:629-641). For example, vancomycin is recommended by the Infectious Disease Society of America (IDSA) treatment guidelines; however, it is associated with a high rate of CDI recurrence and has been shown to have increased resistance due to disruption of the host microbiota. (Isaac S, et al., Short- and long-term effects of oral vancomycin on the human intestinal microbiota. J Antimicrob Chemother 2017; 72:128-36; and Peng Z, et al., Update on antimicrobial resistance in C. difficile: resistance mechanisms and antimicrobial susceptibility testing. (J Clin Microbiol 2017; 55:1998-2008)).

In addition, treatment with antibacterial compounds such as vancomycin may result in decreased microbiome diversity of Firmicutes, Actinobacteria and Bacteroidetes with a characteristic Proteobacteria overgrowth. (Garey K W, et al. A randomized, double-blind, placebo-controlled, single and multiple ascending dose Phase 1 study to determine the safety, pharmacokinetics and food and faecal microbiome effects of ibezapolstat administered orally to healthy subjects. J Antimicrob Chemother 2020; 75 (12): 3635-3643). Proteobacteria overgrowth is associated with a markedly increased risk of systemic infections with MDR Gram-negative microorganisms. Vancomycin for treatment of bacterial infections in the gut is associated with high rates of recurrence and dysbiosis—with approximately 20-25% of patients having a recurring infection after treatment is discontinued. (Gonzales-Luna A J, Carlson T J, Dotson K M, et al. PCR ribotypes of Clostridioides difficile across Texas from 2011 to 2018 including emergence of ribotype 255. Emerg Microbes Infect 2020; 9 (1): 341-7). Thus, new therapies with distinct mechanisms of actions directed against harmful bacterial pathobionts are urgently needed.

In particular, there is a need for compounds having the ability to reduce or eliminate harmful microbial overgrowth in the intestinal milieu that can cause infection or disease while also maintaining the delicate balance of microorganisms that make up the gut microbiome. Such treatments may result in a clinical cure and also provide protection against recurring overgrowth, infection, and/or dysbiosis. Thus, there is also a need for a treatment that promotes microbiome health.

### SUMMARY

The methods and compositions provided herein relate to promoting gut microbiome health by administering low G+C Directed Nucleoside (LDN) Analog to a subject.

Provided herein are methods of promoting gut microbiome health in a subject, comprising: administering an effective amount of a Low G+C Directed Nucleoside (LDN) Analog to a subject suffering from dysbiosis of the gut; wherein the LDN analog simultaneously reduces or eliminates growth of harmful Gram-positive bacteria having low G+C content in the gut microbiome while maintaining and/or increasing beneficial microorganisms in the gut microbiome. In some aspects, the LDN Analog may be a small-molecule inhibitor of DNA pol IIIC enzyme, for example, ibezapolstat. In some aspects, the LDN Analog may target the DNA pol IIIC of low G+C class bacteria whose genomes contain <50% guanine (G)+cytosine (C). In some aspects, the LDN analog may selectively target physiologically harmful species belonging to Streptococcus, Enterococcus, Staphylococcus, Bacillus, Clostridioides, Pneumococcus, Listeria, Mycoplasma, and/or Lactobacillus. In some aspect, the beneficial microorganisms may comprise Firmicutes and include Lachnospiraceae and Lactobacilluseae. In yet other aspects, administering the effective amount of the LDN analog may reduce the growth or prevent regrowth of low G+C content Gram-positive pathobionts in the gut microbiome within 30 days.

Also disclosed herein is a method of achieving and/or maintaining healthy proportions of gut microflora in an intestinal milieu of a subject, comprising: administering an effective amount of a compound directed against DNA pol IIIC enzyme in low G+C content Gram-positive pathobionts to the subject; reducing or eliminating physiologically harmful pathogenic microorganisms belonging to phylum Bacillota; and increasing and/or maintaining physiologically beneficial microorganisms in the intestinal milieu. In some examples, the compound may be a LDN Analog. In some aspects, administering the LDN analog may be prophylactic, the subject may be healthy, and the LDN analog may restore or maintain a mutualistic relationship between the subject and microorganisms in the intestinal milieu. Alternatively, or in addition, in some aspects the subject may be suffering from over-growth of Clostridioides difficile in the gut. In some examples, the physiologically beneficial microorganisms in the intestinal milieu may comprise anaerobic Gram-positive bacterium belonging to genus Clostridium and include C. coccoides.

Also provided herein is a composition for promoting gut microbiome health comprising a Low G+C Directed Nucleoside (LDN) Analog, wherein the LDN analog inhibits DNA pol IIIC enzyme in physiologically deleterious pathobionts, thereby reducing harmful Gram-positive bacteria while allowing beneficial microorganisms to flourish in the gut microbiome. Further, in some aspects, the LDN analog may be a prophylactic treatment and may promote persistence and/or regrowth of healthy microbiota. In some examples, the LDN analog may selectively reduce the growth of pathogenic members of Streptococcus, Enterococcus, Staphylococcus, Bacillus, Clostridioides, Pneumococcus, Listeria, Mycoplasma, and/or Lactobacillus.

### BRIEF DESCRIPTION OF THE DRA WINGS

The patent or application file contains at least one drawing executed in color. Copies of this patent or patent application publication with color drawing(s) will be provided by the Office upon request and payment of the necessary fec.

The drawings described herein are for illustration purposes only and are not intended to limit the scope of the present disclosure in any way.

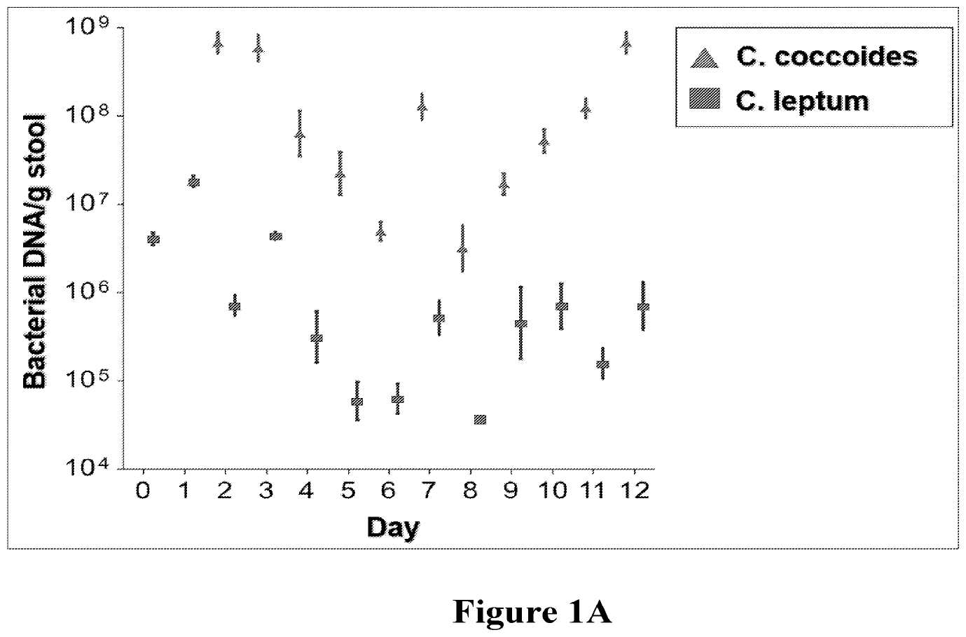

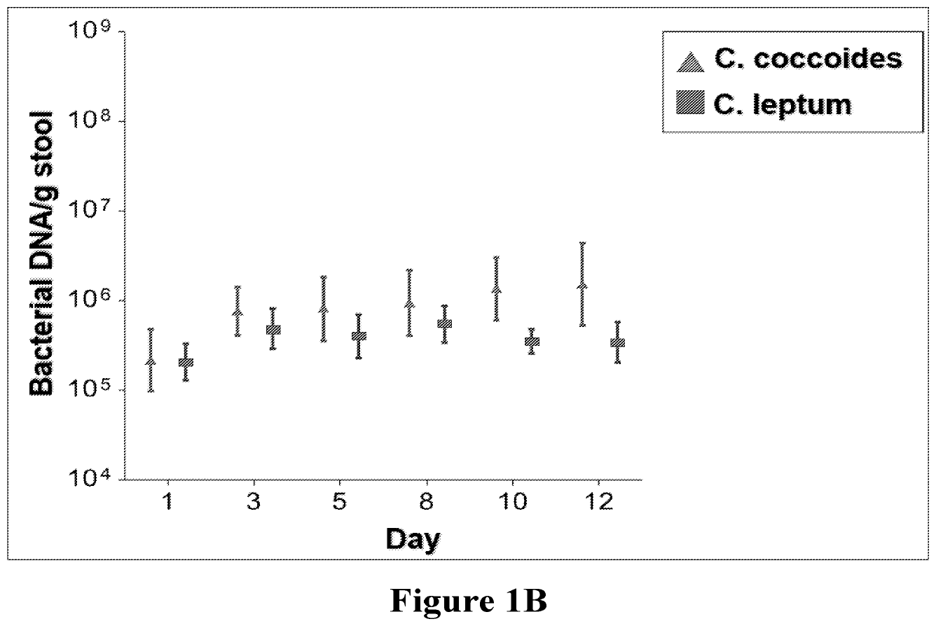

FIG. 1 presents the results of qPCR experiments. FIG. 1A shows quantitation of relevant Firmicutes isolated from samples taken from healthy volunteers in Phase 1; and FIG. 1B shows quantitation of relevant Firmicutes isolated from samples taken from subjects suffering from CDI in Phase 2a;

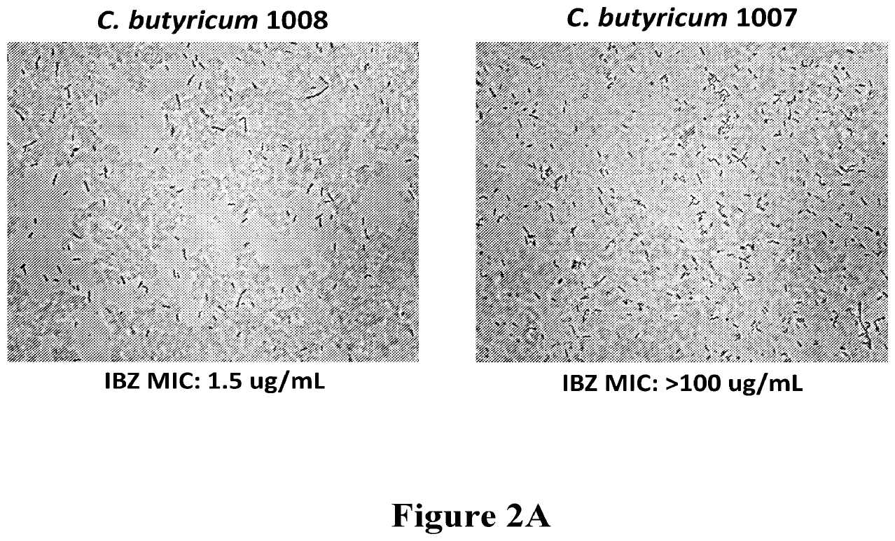

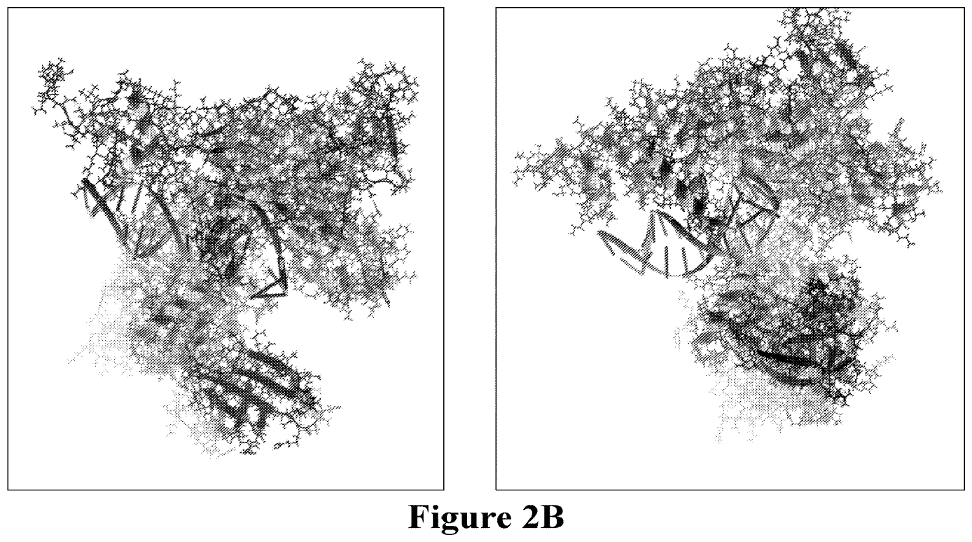

FIG. 2 is a schematic showing Firmicute ibezapolstat (IBZ) susceptibility. FIG. 2A shows the variable susceptibility among Clostridium butyricum strains (C. butyricum 1008 and C. butyricum 1007); and FIG. 2B is a depiction and comparison of the structure of the two C. butyricum strains;

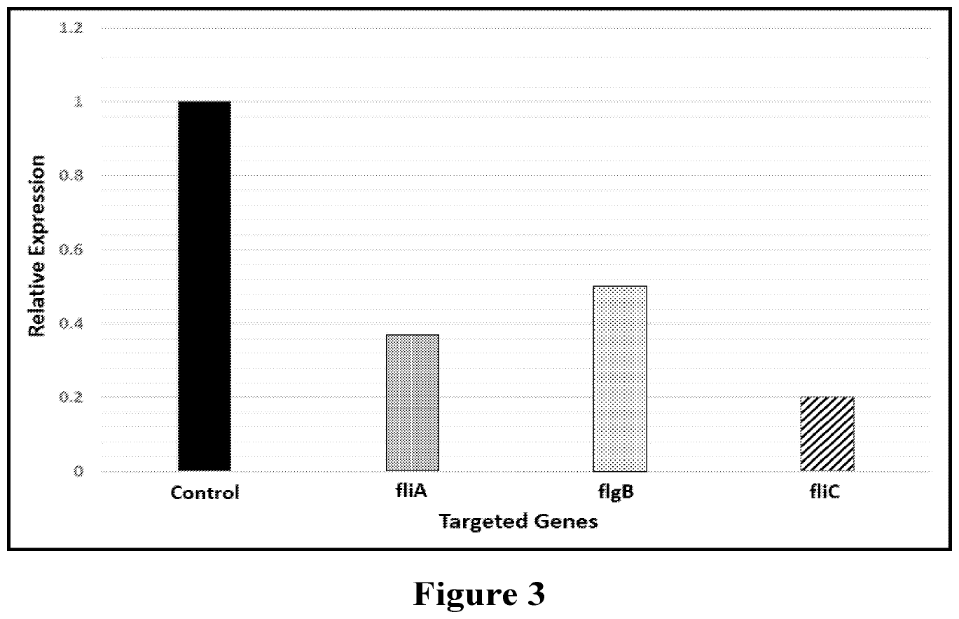

FIG. 3 is a graph showing targeted flagellar genes on the x-axis and relative expression of these genes compared to the non-treated control on the y-axis. In addition, to better visualize the data, the relative expression of the control was normalized to the value 1;

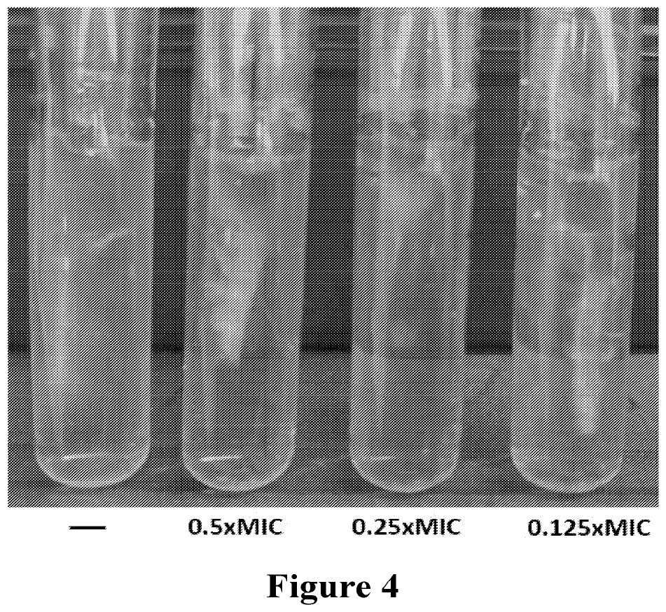

FIG. 4 presents a motility assay comparing the mobility of a control strain of C. difficile to strains obtained from isolates and exposed to sub-inhibitory (not killing) concentrations of ibezapolstat;

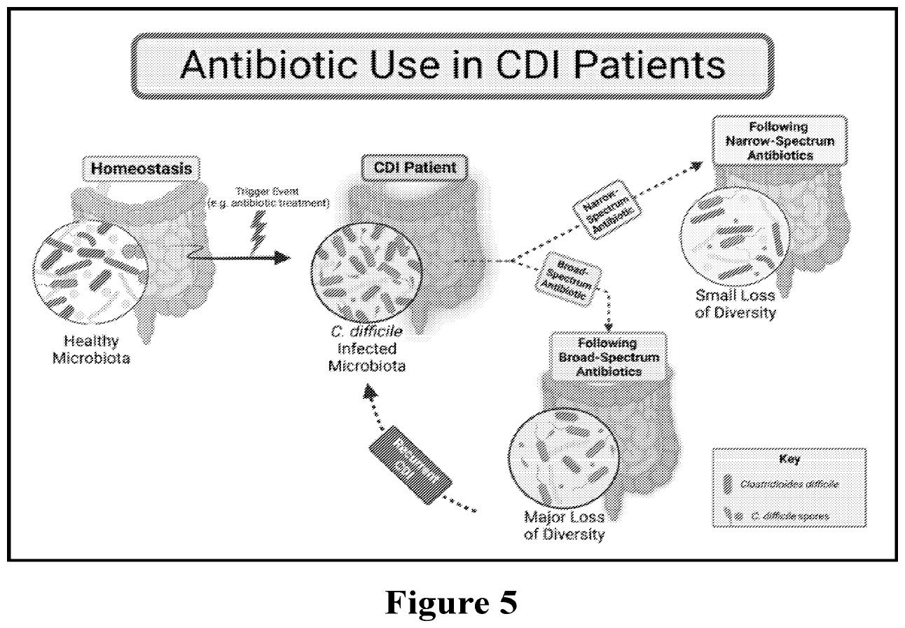

FIG. 5 shows a graphic depicting changes to the gut microbiome following antibiotics in C. difficile infected (CDI) patients and how this can increase the chance of recurrent CDI. The graphic was designed using BioRender;

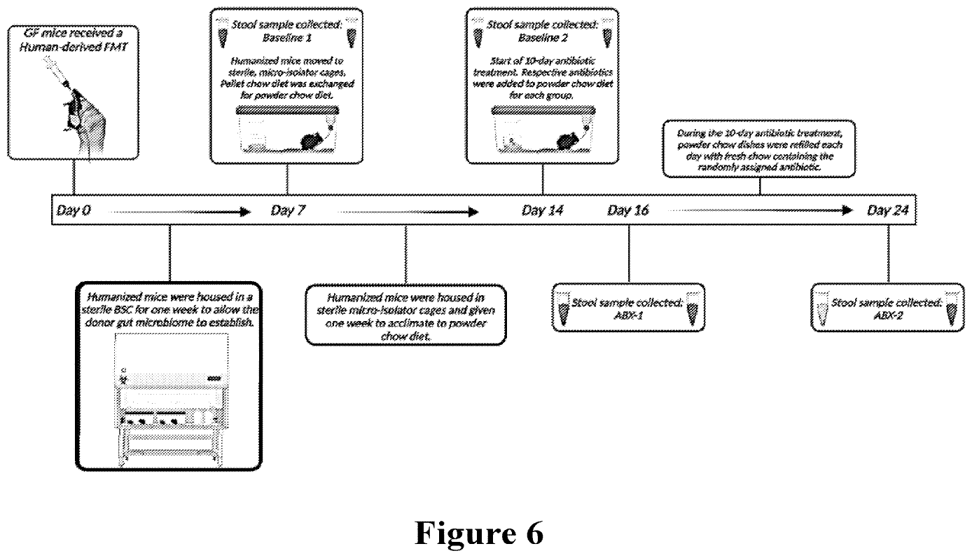

FIG. 6 shows a graphic depicting the germ-free humanized mouse study design. The graphic was created using BioRender;

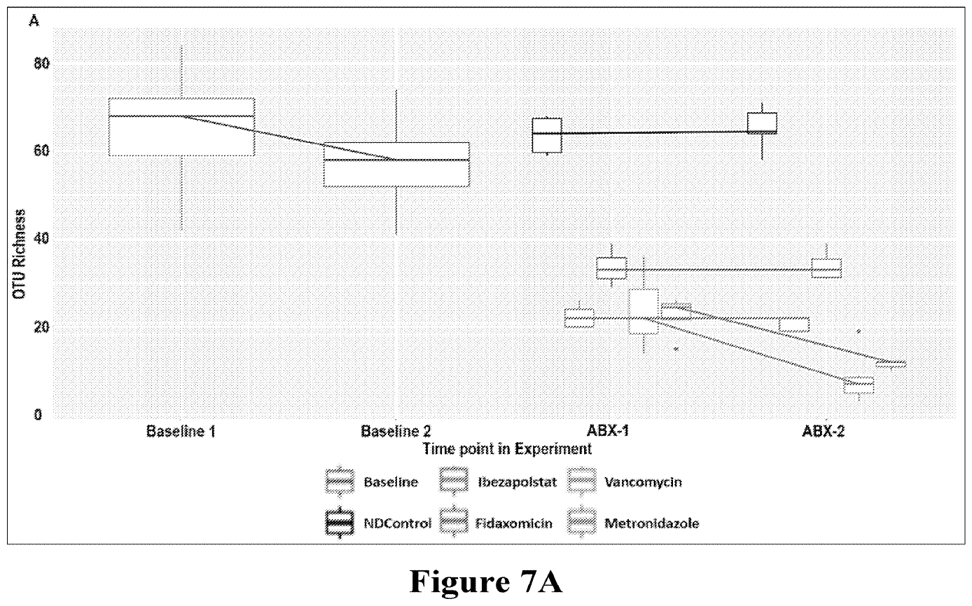

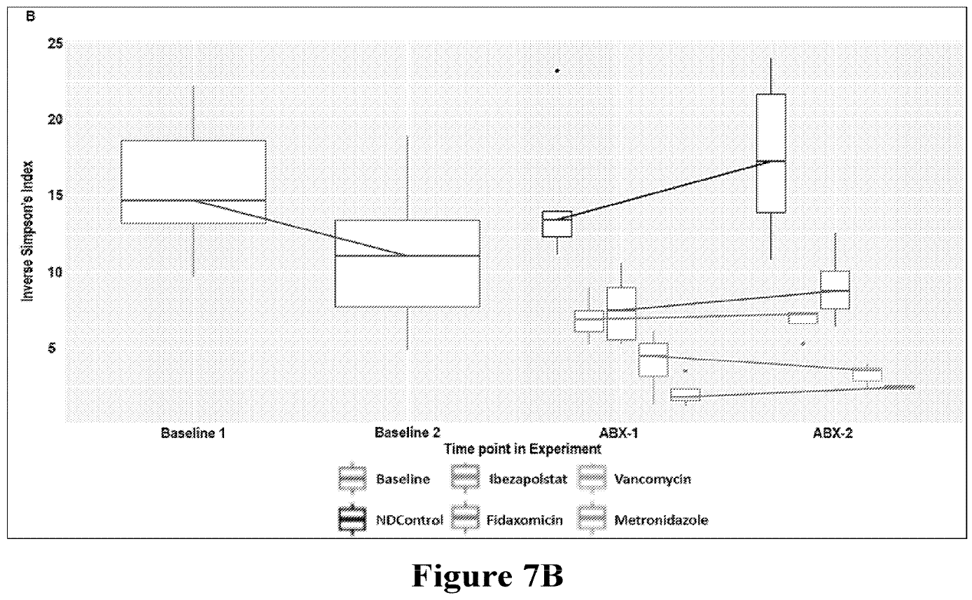

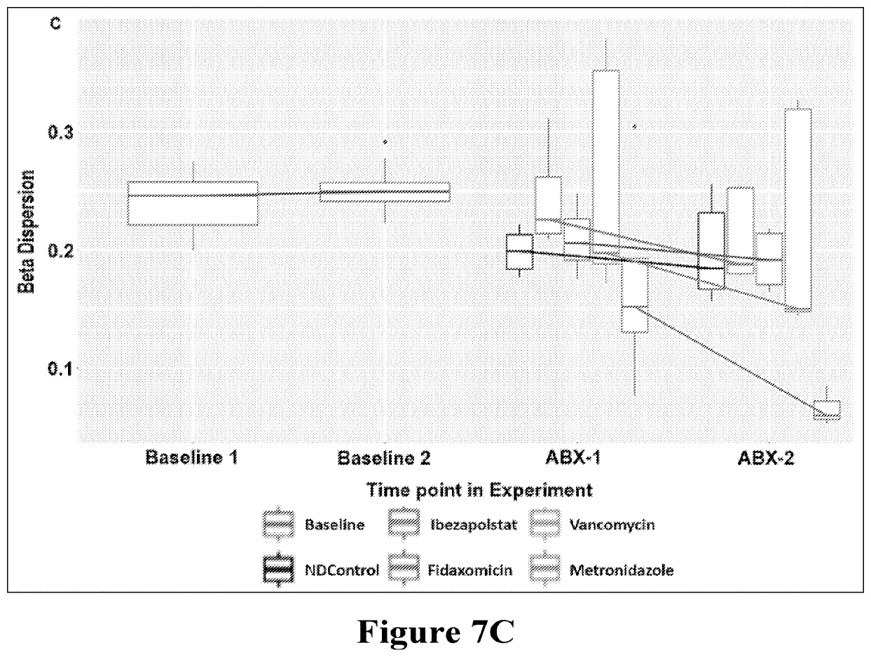

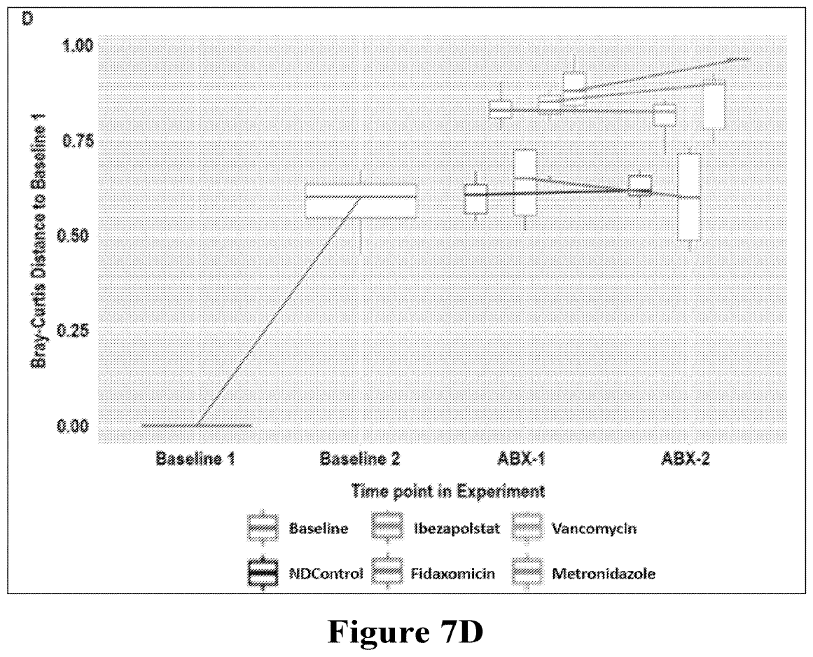

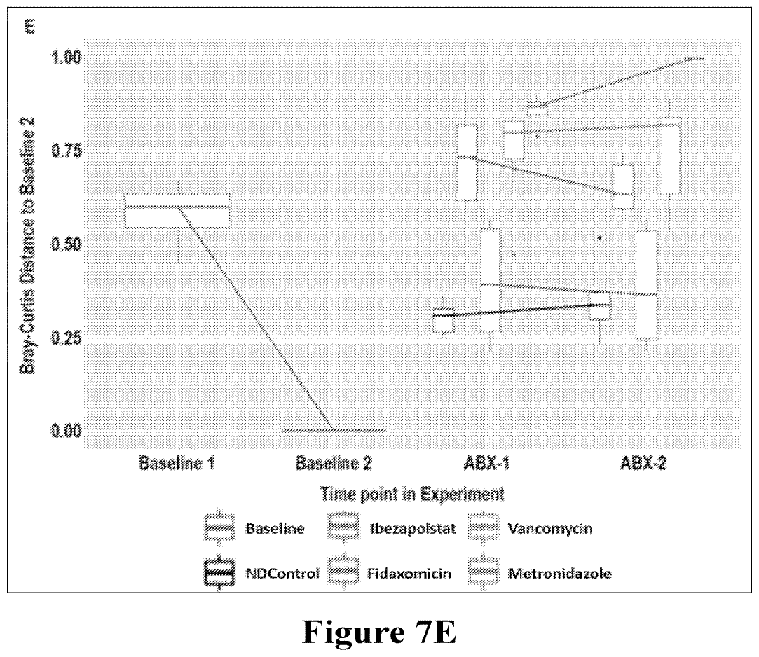

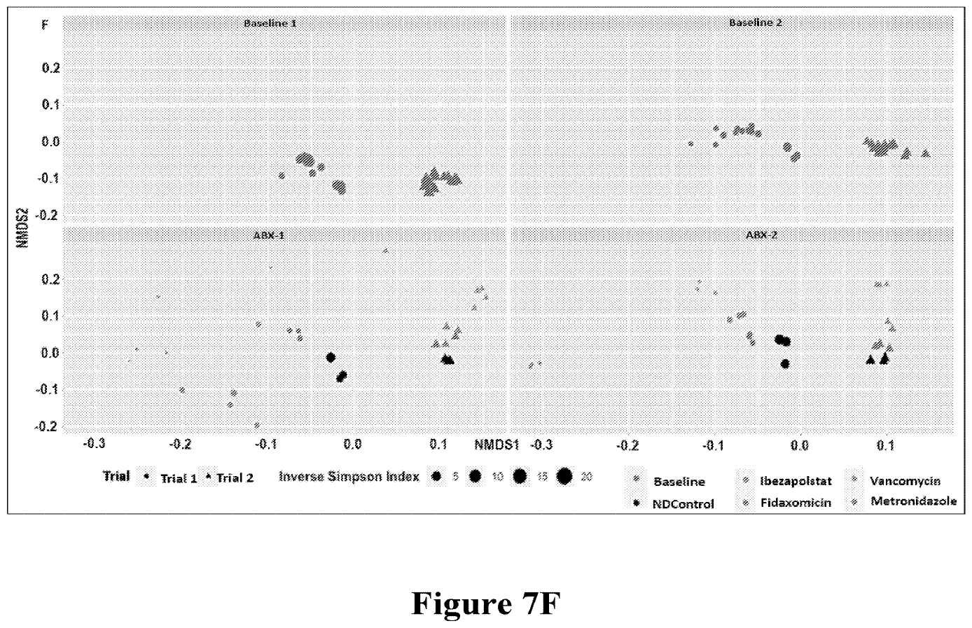

FIG. 7A-E presents box plots with lines connecting groups based on color. Colors represent the antibiotic to which the mice were exposed during the final 10-days of the experiment (or lack thereof in the cases of the no drug control (ND Control) and baseline samples). FIGS. 7A and 7B depict illustrations of the changes in α-diversity of the gut microbiome throughout the experiment. FIG. 7A illustrates the changes in the richness of Operational Taxonomic Units (OTUs) within the gut microbiome over the course of the experiment; and FIG. 7B illustrates the changes in the in the inverse-Simpson's Index within the gut microbiome over the course of the experiment. FIGS. 7C, 7D, and 7E present illustrations of the changes in β-diversity (diversity between groups) of the gut microbiome throughout the experiment. FIG. 7C illustrates the changes in beta dispersion (distance-to-centroid) of the gut microbiome throughout the experiment; FIG. 7D illustrates changes in Bray-Curtis Dissimilarity to Baseline 1 (D7) throughout the experiment; FIG. 7E illustrates changes in Bray-Curtis Dissimilarity to Baseline 2 (D14); and FIG. 7F shows non-metric multidimensional scaling (NMDS) of Bray-Curtis dissimilarity. Each dot represents one sample taken at the respective time point (labeled at the top of each plot). Size indicates the inverse Simpson Index value for that sample, the shape indicates from which trial the sample was collected and the color indicates which antibiotic (or lack thereof) each sample was exposed to;

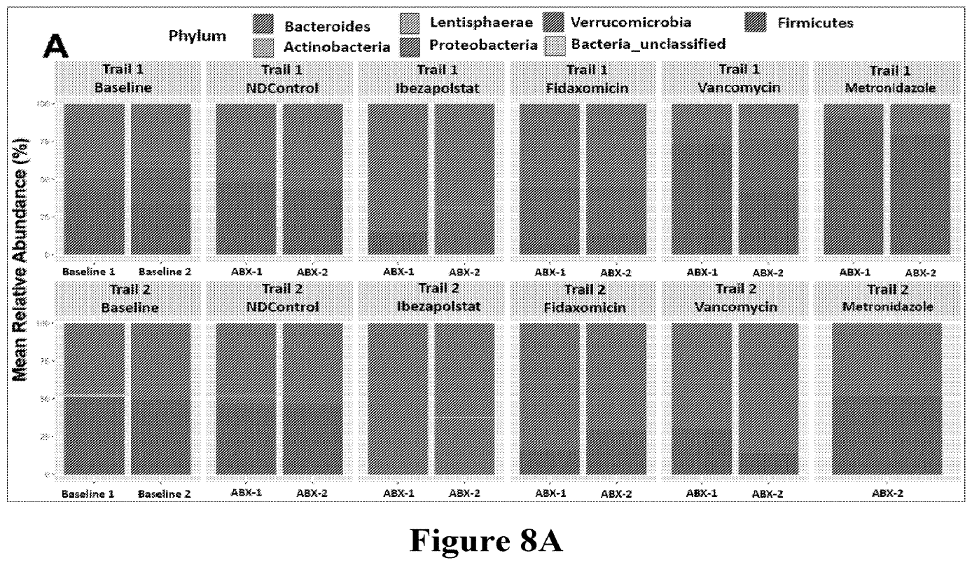

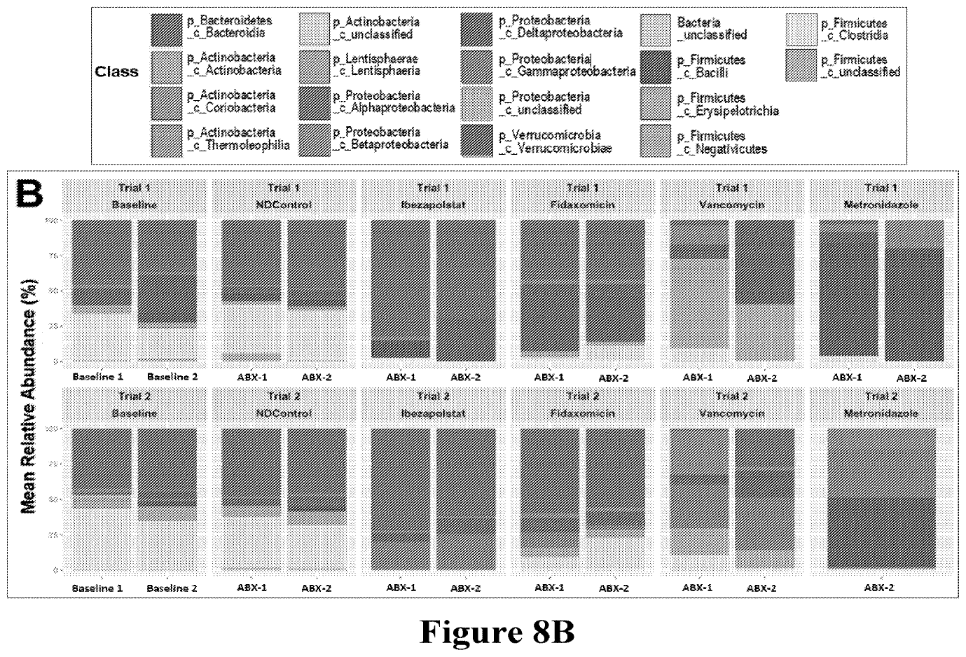

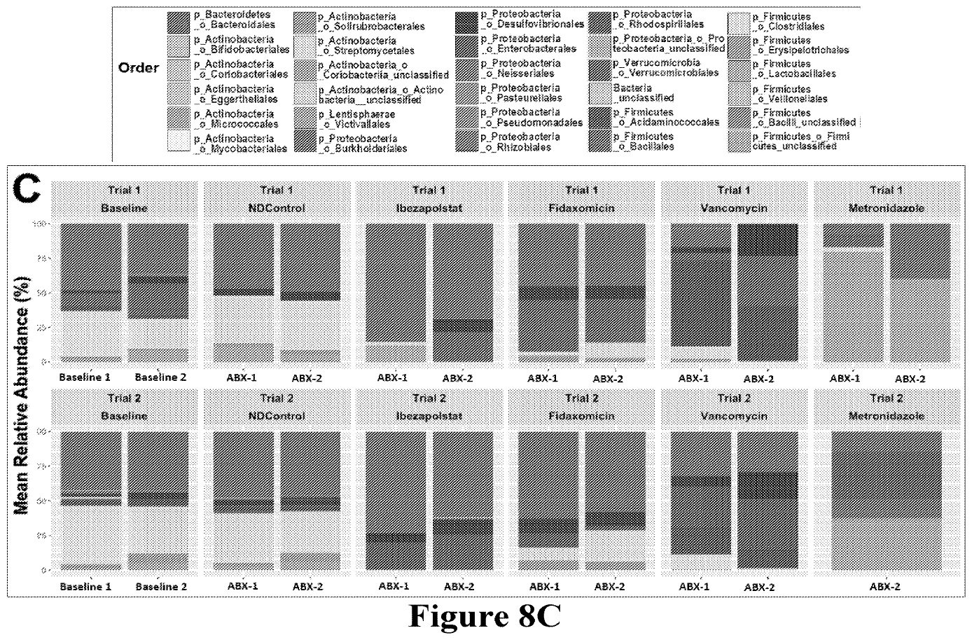

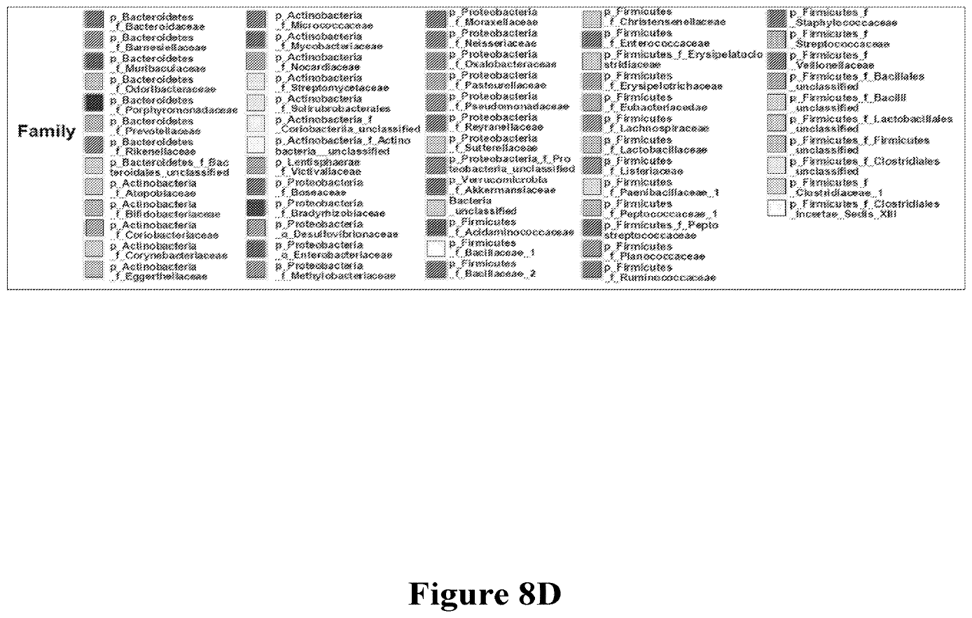

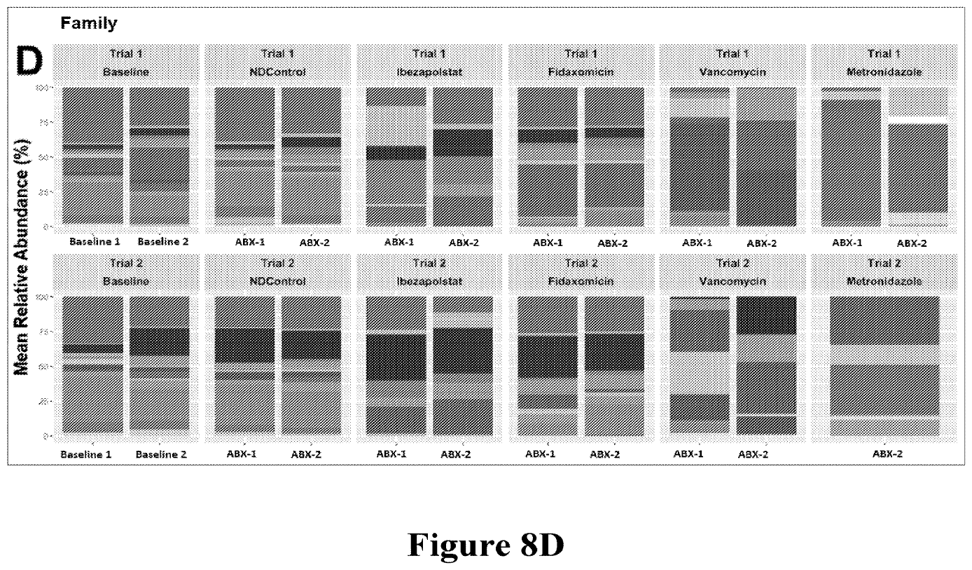

FIG. 8A-D shows stacked bar charts illustrating the mean relative abundance (represented as a percentage) of different bacterial taxonomy levels throughout the experiment. FIG. 8A represents the phylum level, FIG. 8B represents the class level, FIG. 8C represents the order level, and FIG. 8D represents the family level;

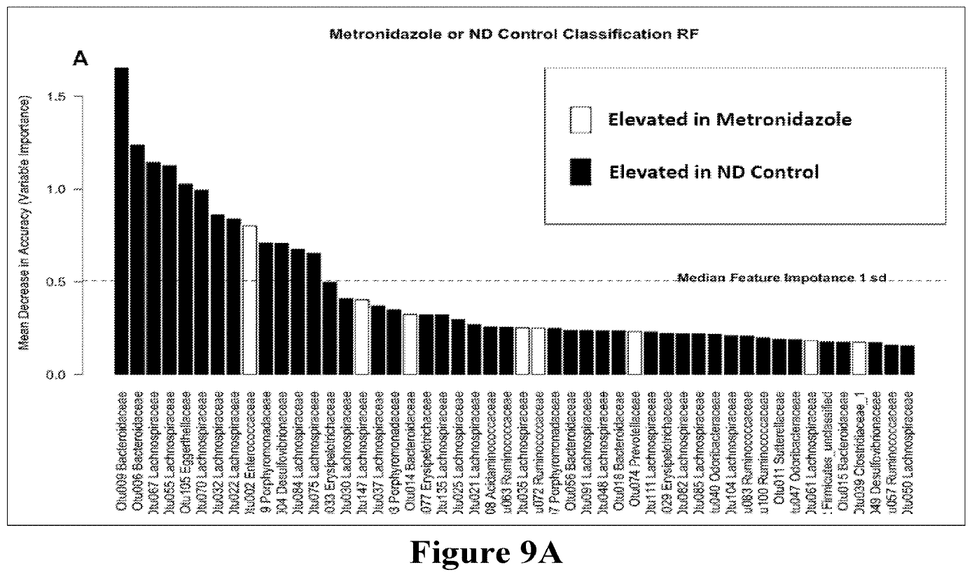

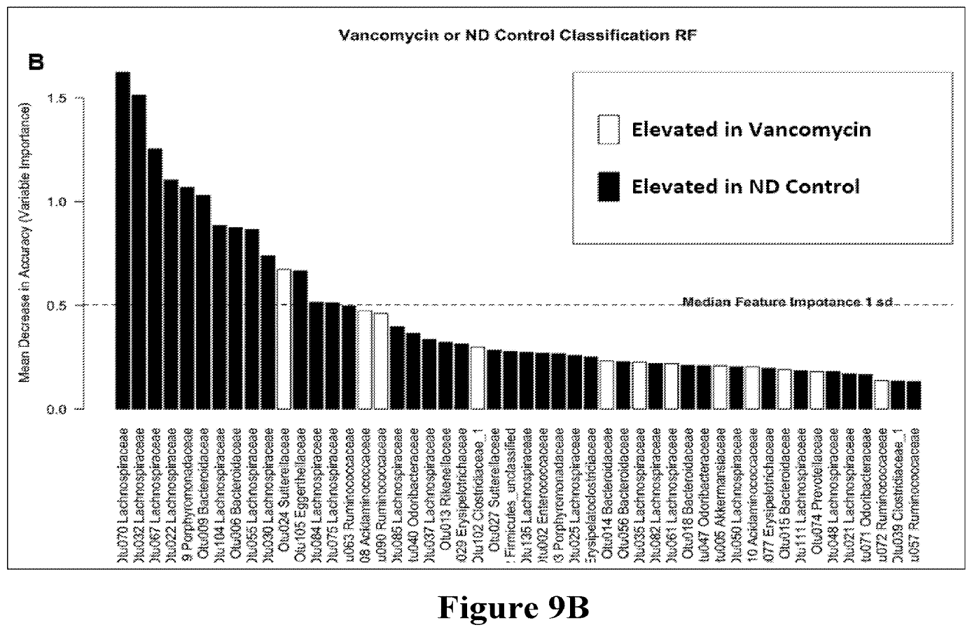

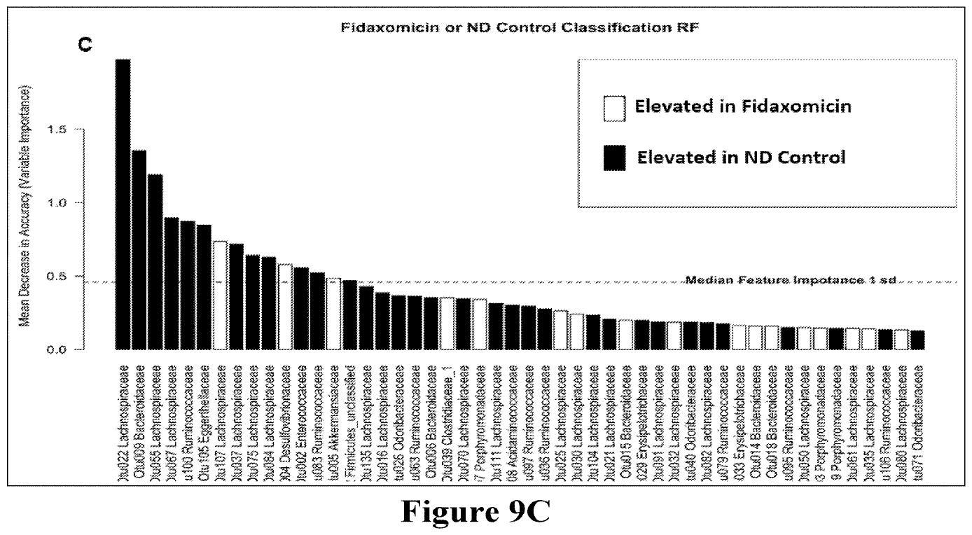

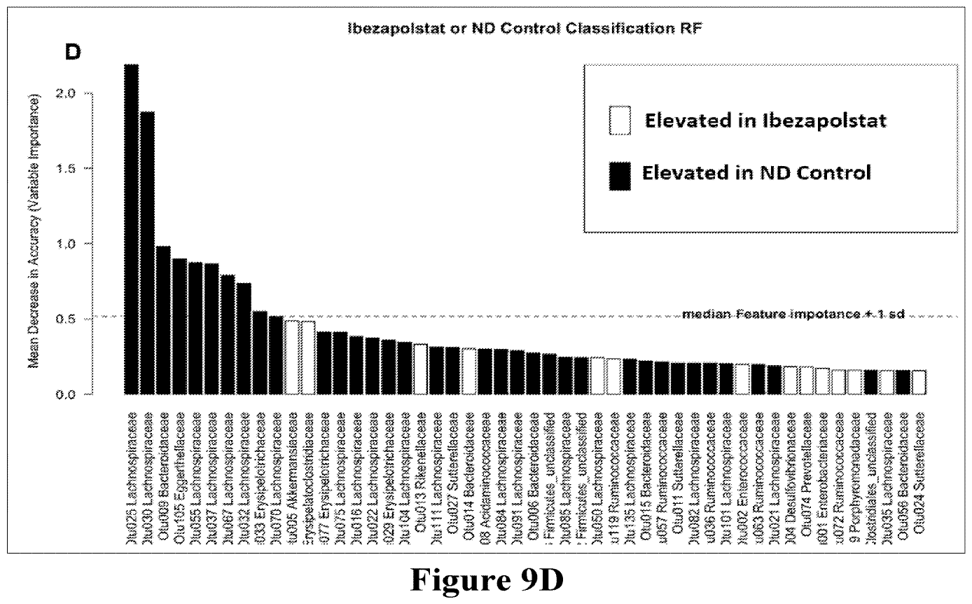

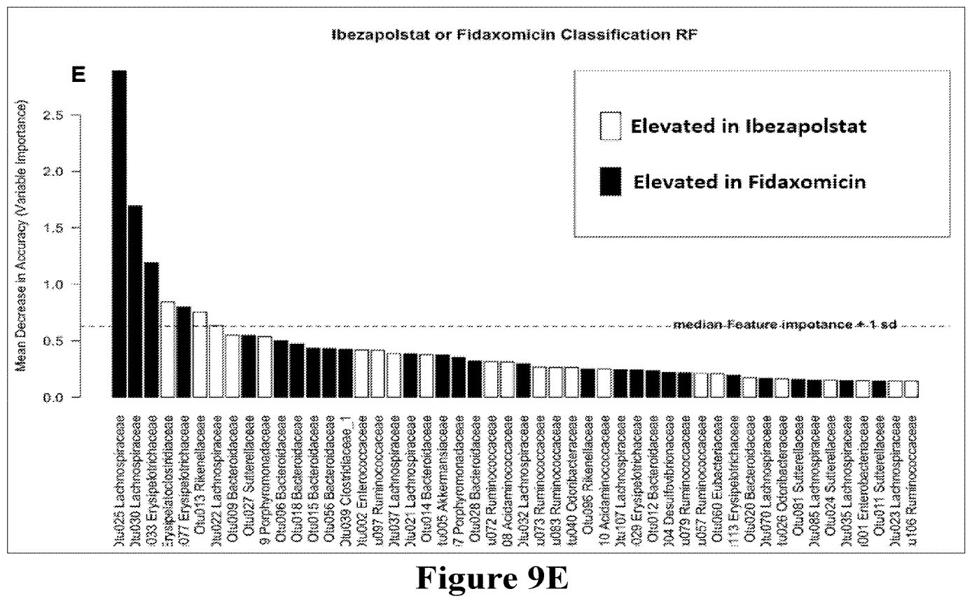

FIG. 9A-D present bar charts of OTUs identified by random forest analysis that distinguish specified treatment groups. The dashed line represents the significance cut-off based on the median importance level given 1 standard deviation in both directions and OTUs shown (x-axis) are classified to the family level. Comparisons shown in legend and enriched (elevated) OTUs are colored for each panel. FIGS. 9A-D show comparisons of each group of mice exposed to an antibiotic or the no drug control group (ND Control). FIG. 9E compares mice exposed to ibezapolstat to mice exposed to fidaxomicin;

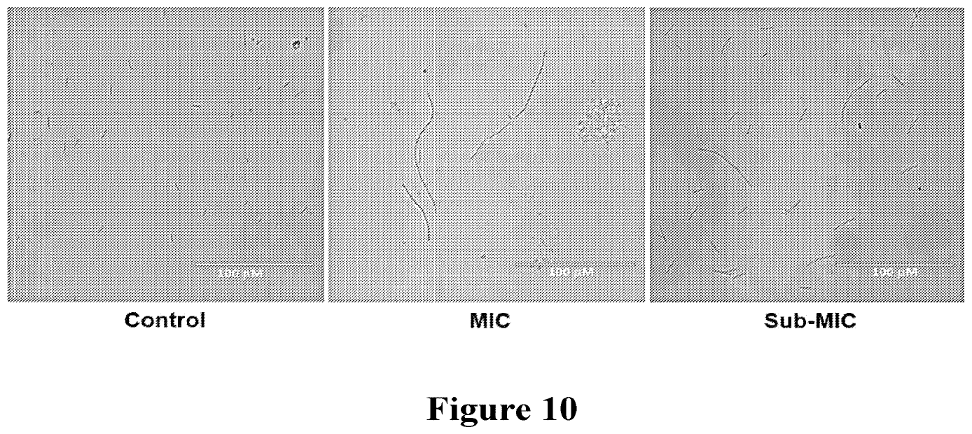

FIG. 10 shows Ibezapolstats effect on the morphology of C. difficile strain CD 630;

FIG. 11 shows the effects of Ibezapolstat on CD 630 motility;

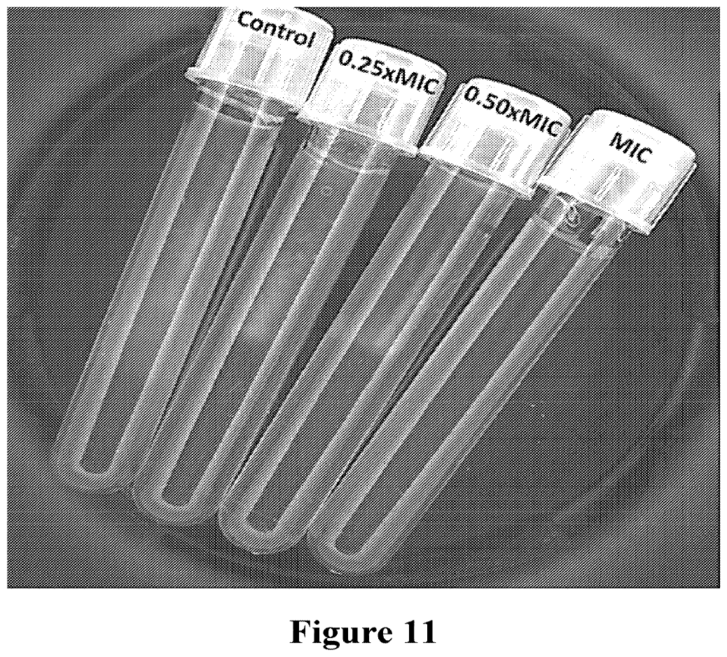

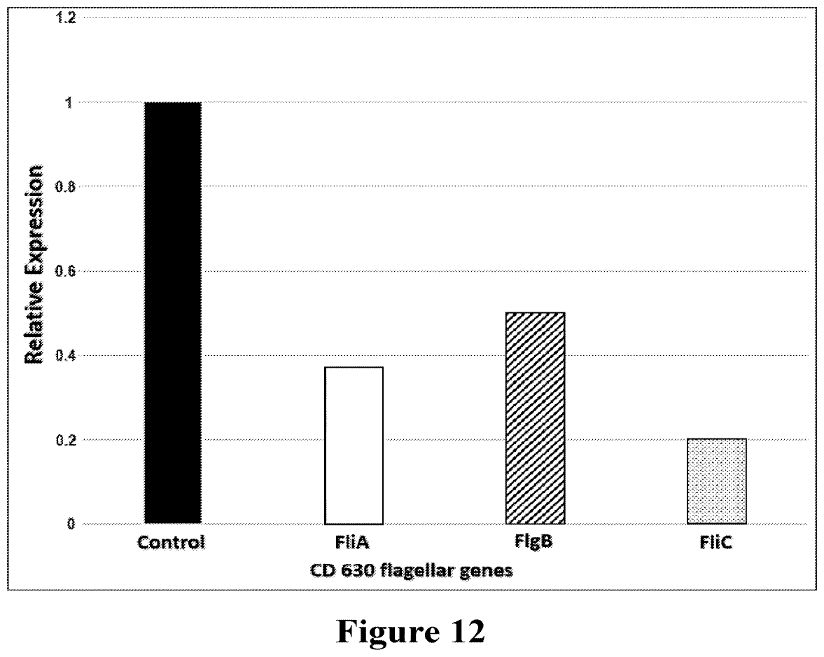

FIG. 12 shows the effects of Ibezapolstat on flagellar gene expression;

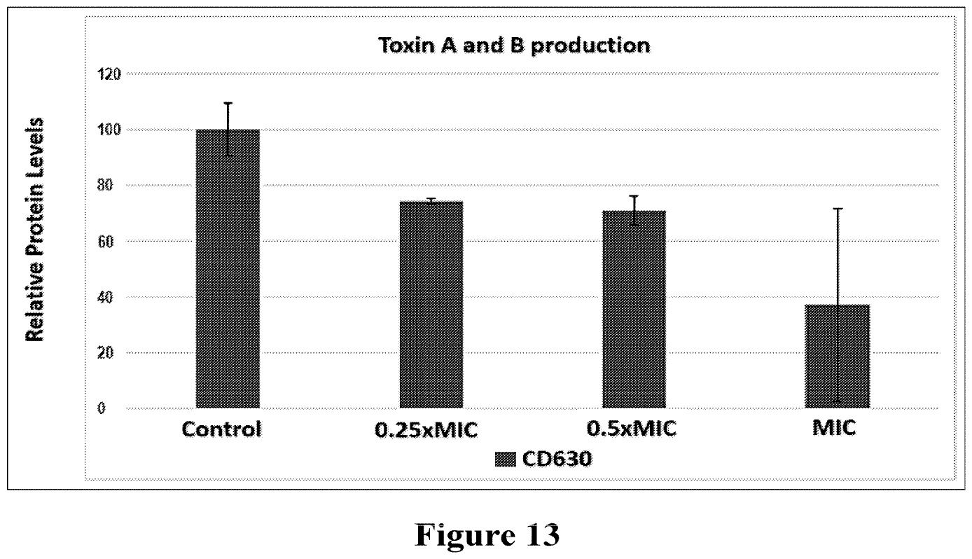

FIG. 13 shows the effects of Ibezapolstat exerts on modulating C. difficile toxin production;

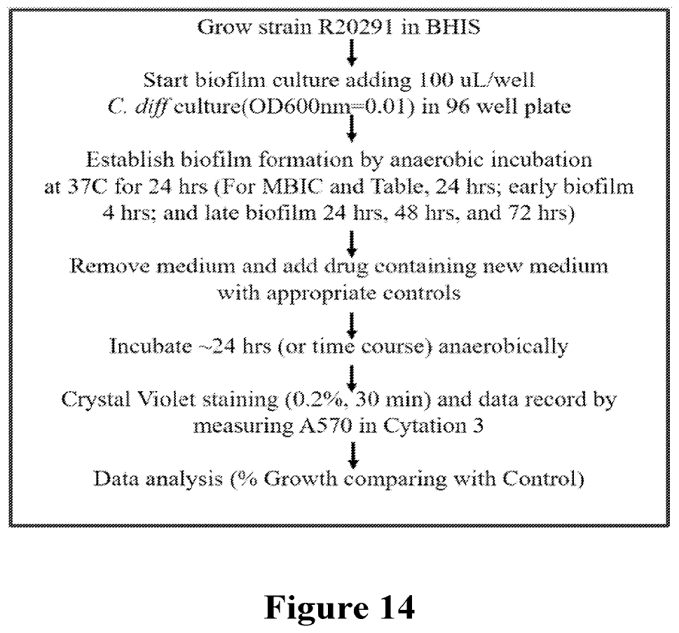

FIG. 14 presents basic flow chart outlining the experimental procedure used in these studies may be seen in;

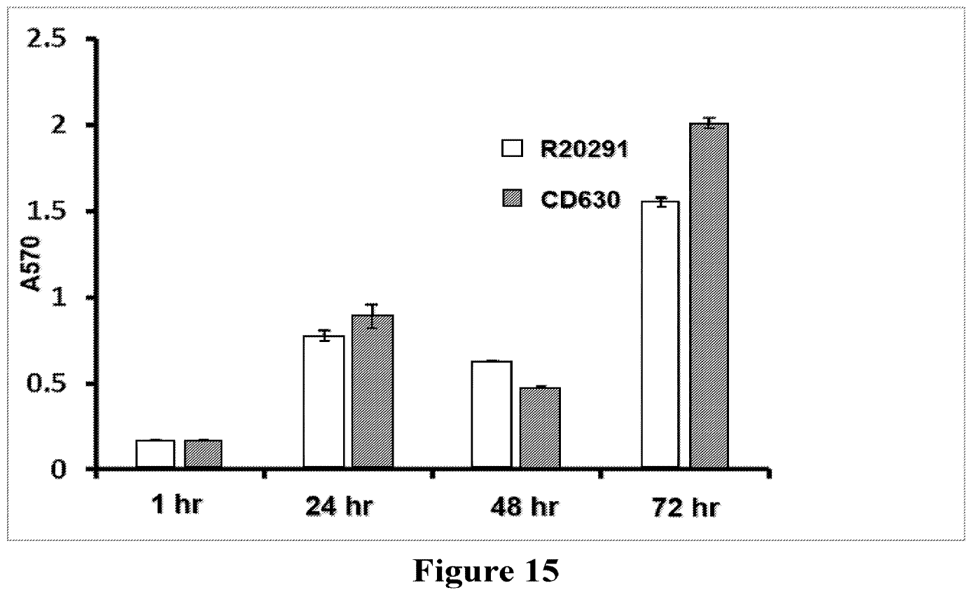

FIG. 15 shows biofilm formation by C. difficile laboratory strains R20291 and CD630;

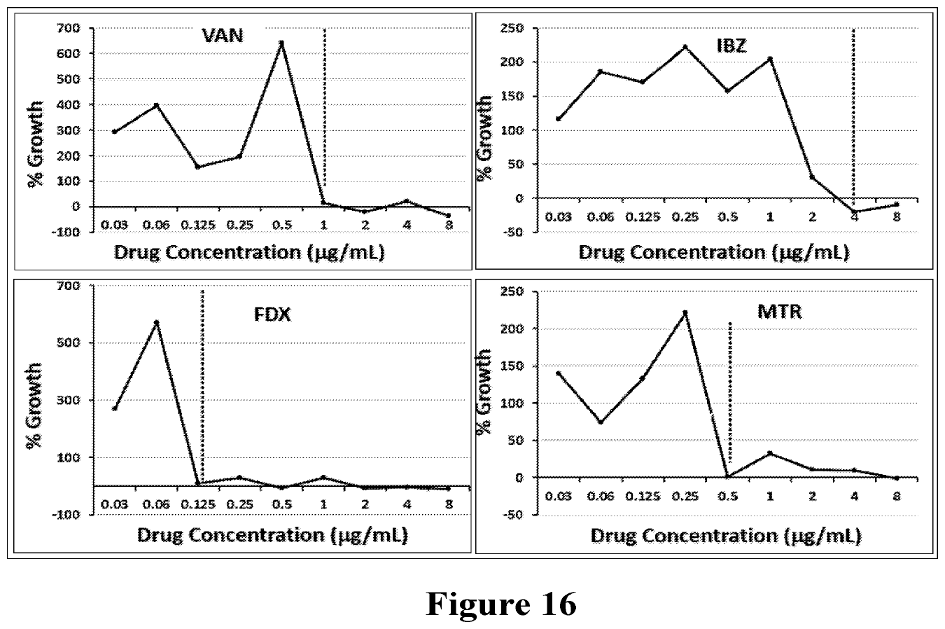

FIG. 16 presents Minimum Biofilm Inhibitory Concentration (MBIC) and the Eagle effect corresponding to antibiotic treatment with IBZ, vancomycin (VAN), fidaxomicin (FDX), or metronidazole (MTZ);

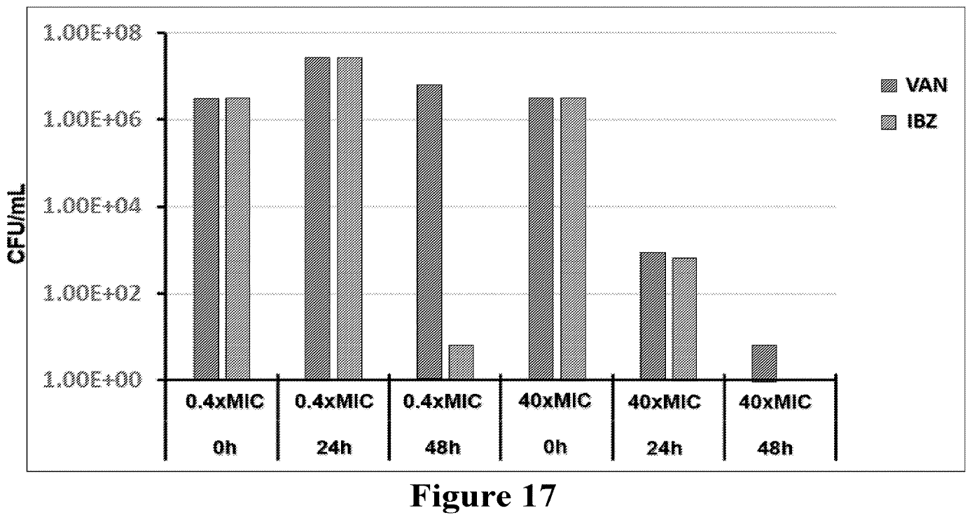

FIG. 17 is a graph that demonstrates the effect of MIC or sub-MIC levels of IBZ and VAN on early (4 hrs) biofilms by measuring CFU/mL over a period of 24 or 48 hours;

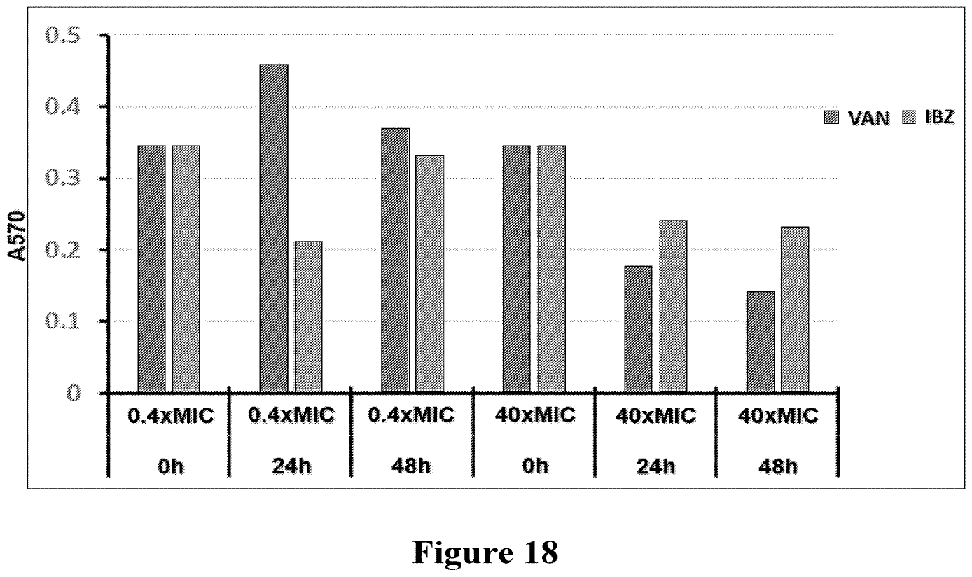

FIG. 18 is a graph that demonstrates the effect of MIC or sub-MIC levels of IBZ and VAN on early (4 hrs) biofilms by measuring optical density at A570 over a period of 24 or 48 hours; and

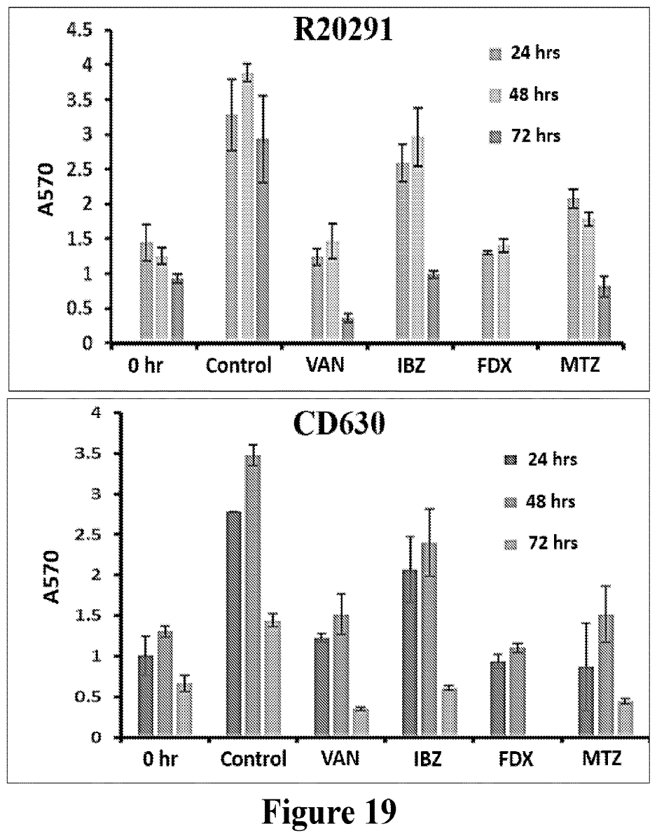

FIG. 19 shows the effects of IBZ and comparable antibiotics on early (24 hrs) biofilms comprising either C. difficile laboratory strains R20291 or CD630 and measuring optical density at A570 over a period of 24 or 48 hours.

### DETAILED DESCRIPTION

The features and other details of the invention will now be more particularly described. It will be understood that particular embodiments described herein are shown by way of illustration and not as limitation of the invention. The principle feature of this invention can be employed in various embodiments without departing from the scope of the invention.

Described herein are methods and compositions for promoting gut microbiome health via administering a Low G+C Directed Nucleoside (LDN) Analog, for example an inhibitor of DNA pol IIIC enzyme to a subject. The LDN analog described herein may be directed to low G+C class bacteria whose genomes contain <50% guanine (G)+cytosine (C). Further, the LDN analog provided may selectively inhibit physiologically harmful pathobionts belonging to the Bacillota phylum, which may include members of Firmicute or Bacillales. Additionally, the LDN analog may inhibit the proliferation or replication of pathobionts from the genus Clostridium, for example, C. difficile while also promoting the growth of beneficial members of the Clostridium genus, for example, C. coccoides. In some examples, the LDN analog provided may selectively target Gram-positive members of Bacillales including members of the Staphylococcaceae family and/or Enterococcus, for example, S. aureus and E. faecium. Further, the LDN analog may simultaneously facilitate persistence or regrowth of healthy microbiota in the intestinal milieu. For example, growth of Actinobacteria may increase in the gut microbiome of a subject with the administration of the LDN analog provided. In some aspects, the LDN analog may selectively target at least one single nucleotide polymorphism (SNP) in healthy microbiota organisms allowing continued growth of these healthy organisms in the presence of the LDN.

Also described herein are methods and compositions that may target flagellar genes and thus result in a decrease in motility in gram-positive organisms. For example, the compositions provided may result in a reduction in flagellar gene expression, including common flagella-associated genes fliA, flgB, fliC-VIP.

Ibezapolstat (IBZ) is a non-absorbable antimicrobial for the treatment of C. difficile infection (CDI). In vitro and human studies have shown potent activity of IBZ against C. difficile but selective activity against other beneficial Gram-positive gut microbiota shown to reduce the risk of dysbiosis. Although the target DNA pol IIIC enzyme is present in most Gram-positive species, IBZ susceptibility demonstrates selectivity among certain Gram-positive species that may be found in gut microbiota.

### Definitions

By Low G+C Directed Nucleoside (LDN) Analog it is meant any molecule and/or compound in a class of nucleoside-analog inhibitors that may selectively target and inhibit the DNA polymerase IIIC (DNA pol IIIC) enzyme of Gram-positive microorganisms having low G+C (bacteria with genomes comprise fewer Guanine and Cytosine nucleotide bases as opposed to Adenine and Thymine/Uracil bases) content, such as Firmicutes and Bacillales having low G+C content. Such molecules may include but are not limited to 7-substituted-N2-(3,4-dichlorobenzyl) guanines (DCBGs) and may selectively inhibit the DNA pol IIIC. Such molecules may also be 1,7-dihydro-6H-purin-6-one compounds, such as those compounds in U.S. Pat. Nos. 6,926,763 and 8,796,292 incorporated by reference herein.

For example, Ibezapolstat is 2-((3,4-dichlorobenzyl)amino)-7-(2-morpholinoethyl)-1,7-dihydro-6H-purin-6-one.

By “effective amount” is meant an amount sufficient to effect beneficial or desired clinical or biochemical results. An effective amount can be administered one or more times. For example, an effective amount may be the amount which when administered to a site of infection or potential infection will treat or prevent a bacterial infection, while simultaneously increasing the amount and/or proportion of Actinobacteria and/or Firmicutes in the microbiome.

By “selective target” or “selectively targeting” it is meant a mechanism that is specifically directed to inhibit the DNA pol IIIC enzyme present in Gram-positive microorganisms with a genome having low amounts of G+C (fewer guanine and cytosine nucleotide based in the genome than adenine and thymine/uracil bases).

By “administration” or “administering” is meant a method of giving one or more unit doses of an LDN analog, such as ibezapolstat to an animal, e.g., a mammal (such as topical, oral, intravenous, intraperitoneal, or intramuscular administration). The method of administration may vary depending on various factors, e.g., the components of the pharmaceutical composition, site of the potential or actual infection, and severity of the actual microbial infection.

By “inhibiting” is meant reducing the cellular growth rate of a bacterium by at least 80%. In certain embodiments, the growth can be inhibited by 90%, 95%, or even 99% or more. The degree of inhibition can be ascertained, for example, by an in vitro growth assay, e.g., by a standard liquid culture technique. Inhibition of colony formation at suitable MICs (minimal inhibitory concentrations), e.g., <100 μg/ml, more preferably <10 μg/ml, are preferred.

By “treatment” is meant an approach for obtaining beneficial or desired clinical results. For the purposes of this invention, beneficial or desired clinical results include, but are not limited to, alleviation of symptoms, diminishment of extent of disease, stabilization (i.e., not worsening) of a state of disease, delay or slowing of disease progression, amelioration or palliation of the disease state, and remission (whether partial or total), whether detectable or undetectable. “Treatment” refers to both therapeutic treatment and prophylactic or preventative measures. Those in need of treatment include those already with the disorder as well as those in which the disorder is to be prevented and/or recurrence is to be prevented. “Palliating” a disease means that the extent and/or undesirable clinical manifestations of a disease state are lessened and/or the time course. of the progression is slowed or lengthened, as compared to a situation without treatment.

By “microbiome” is meant the microorganisms in a particular environment (including the body or a part of the body). Preferably, the microbiome is located in the gut.

By “intestinal milieu” it is meant the internal environment of the intestinal system which may harbor gut microbiota, and include a complex community of bacteria, archaea, fungi, viruses and protozoans that bring to the host organism an endowment of cells and genes more numerous than its own.

A “healthy microbiome” could be described in terms of ecologic stability (i.e., ability to resist community structure change under stress or to rapidly return to baseline following a stress-related change), by an idealized (presumably health-associated) composition or by a desirable functional profile (including metabolic and trophic provisions to the host). A healthy adult microbiome may also be characterized by a majority of bacterial species in the Firmicute or Bacteroidetes phylum and a minority in the Actinobacteria and Proteobacteria phylum. A healthy newborn microbiome may be characterized by a majority of bacterial species in the Bacteroidetes and Actinobacteria phylum.

By “improving the health of a gut microbiome” is meant that the composition of the microbiome is brought to a majority proportion of bacterial species from the Actinobacteria, Firmicute or Bacteroidetes phylum with a minority of Proteobacteria phylum. Alternatively, improving the health of a gut microbiome can mean increasing the proportion of bacterial species in the Actinobacteria phylum, such as is present in a healthy newborn gut microbiome. A subject may be suffering from a pathogenic bacterial infection or not suffering from such an infection.

By “reducing the likelihood of infection” is meant prophylactic treatment or treatment resulting in a reduction (e.g., at least 5%, 10%, 15%, 20%, 25%, 30%, 35%, 40%, 50%, 60%, 70%, 80%, 90%, or 95%) for a subject or a patient population in the chance or rate of developing a microbial infection by administering a compound compared to a subject or patient population not receiving the LDN analog.

By “clinical cure” is meant the initial infection has cleared. It is preferably measured about 10-12 days after diagnosis after a subject has received a treatment course.

By “sustained clinical cure” is mean the subject had a clinical cure and the infection did not recur. It is measured at 30-90 days after diagnosis.

By “recurrence” is meant that the subject had a clinical cure and the infection occurred again within 30-90 days' time.

By “animal” is meant any animal susceptible to a Gram-positive bacterial infection. Such as animal may include humans, dogs, cats, pigs, cows, horses, goats, chickens, turkeys, sheep, rats, mice, and rabbits, as well as other animals kept for commercial purposes or as pets. By an “animal susceptible to a microbial infection” is defined as an animal that is at increased risk, relative to the general population, of contracting a microbial infection. Examples of such animals include those that have recently undergone a surgical procedure, or immunocompromised humans, e.g., those with AIDS (acquired immunodeficiency syndrome) or those having transplants for which immunosuppressive drugs are required. Such animals can be identified using methods known to one of ordinary skill in the art.

By “coating agent” is defined as a biocompatible compound or mixture of stop compounds suitable for coating a surface. Suitable coating agents are known in the art. Exemplary coating agents include, but are not limited to, polymers, e.g., polyethylene glycol, hypromellose, hydroxypropyl cellulose, polytetrafluoroethylene, methylcellulose, polyvinyl alcohol or other polymers that are biocompatible.

By “medium” is defined as any substance, liquid or solid, on which or in which a microbe may be present or in which prevention of the presence of a microbe is desired. Exemplary media include culture media (e.g., agar or broth), food, medical supplies (e.g., sterile fluids), medical devices (e.g., catheters), countertops, and other surfaces.

By “microbial infection” is defined as the invasion of a host animal by pathogenic microbe. For example, the infection may include the excessive growth of a microbe that is normally present in or on the body of an animal or growth of a microbe that is not normally present in or on the animal. More generally, a microbial infection can be any situation in which the presence of a microbial population(s) is damaging to a host animal. Thus, an animal is “suffering” from a microbial infection when an excessive amount of a microbial population is present in or on the animal's body, or when the presence of a microbial population(s) is damaging the cells or other tissue of the animal. In one embodiment, the number of a particular genus or species of microbe is at least 2, 4, 6, or 8 times the number normally found in the animal. Examples of microbes include, but are not limited to, Gram-positive or any other class of bacteria.

By “pharmaceutically acceptable salts” are meant those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methane, sulfonic, formic, benzoic, malonic, naphthalene-2-sulfonic and benzenesulfonic acids. Other acids such as oxalic, while not in themselves pharmaceutically acceptable, may be useful as intermediates in obtaining the compounds of the invention and their pharmaceutically acceptable acid addition salts. Salts derived from appropriate bases include alkali metal (e.g., sodium or potassium), alkaline earth metal (e.g. magnesium), ammonium and NR4⁺ (where R is C₁₋₄ alkyl) salts. Preferred salts include hydrochlorides, hydrobromides, sulfates, mesylates, maleates, tartrates, and fumarates. References hereinafter to a compound according to the invention include compounds of the general formulae shown, as well as their pharmaceutically acceptable salts.

By “prevention” of microbial growth or infection is defined as the application of a compound of the invention such that microbial growth or infection does not occur. The amount of a compound of the invention necessary for prevention of microbial growth can be ascertained, for example, by an in vitro growth assay, e.g., by a standard liquid culture technique. The amount of a compound of the invention necessary for the prevention of microbial infection may be ascertained, for example, by an in vivo assay, e.g., by determining the amount of compound that must be administered in order to prevent infection in a study animal, e.g., a guinea pig, after inoculation with a microbe. In general, compounds showing prevention at suitable concentrations, e.g., <100 μg/ml, more preferably <10 μg/ml, are useful for further examination as therapeutic agents.

By “treating” is defined as the medical management of a patient with the intent that a cure, amelioration, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

By “therapeutically effective amount” is defined as an amount which, when administered to an animal in need, will alleviate at least some of the symptoms of a bacterial infection. In the context of prophylaxis, a “therapeutically effective amount” is an amount which, when administered to an animal susceptible to bacterial infection, will help inhibit or otherwise reduce the likelihood of such an infection.

By “prophylactic” or “prophylactic treatment” is meant administering an effective amount of the LDN analog to a subject that may be considered healthy, that is, the subject is not exhibiting signs or symptoms of disease or dysbiosis in the gut. The subject may or may not be susceptible to dysbiosis in the gut and/or infection. For example, the LDN analog may be administered to the subject to maintain the microbial balance in a healthy gut, promote the mutualistic relationship between the host gut and the microorganisms therein, and/or reduce the growth, replication, or proliferation of physiologically harmful pathobionts such that physiologically beneficial bacteria may flourish.

The details of one or more embodiments of the invention are set forth in the accompanying description below. Other features, objects, and advantages of the invention will be apparent from the description and from the claims.

The microbiome of the healthy gut is composed of major bacterial groups called phyla. Firmicutes (Gram-positive spore forming organisms) and Bacteroidetes (Gram-negative non-spore forming organism) are most common and together generally comprise greater than 90% of the healthy adult gut microbiome. The adult gut microbiome also contains Actinobacteria, Fusobacteria, Verrucomicrobia, and Proteobacteria. Actinobacteria are present in large proportion in children and generally decrease in overall proportion with age (replaced by Firmicutes and Actinobacteria). Proteobacteria (Gram-negative facultative anacrobes) generally comprise 2-5% of the healthy microbiome. When the composition of the microbiome is altered from its normal diversity, the normal physiological functions are disrupted—called dysbiosis. Patients suffering from bacterial infection, for example C. difficile infection, are in a state of dysbiosis. Typically, dysbiosis associated with subjects suffering from bacterial infection includes an increased proportion of Proteobacteria (often referred to as a “bloom”) and a reduced number of Firmicutes and Bacteroidetes.

It has been found that certain bacteria, such as Firmicutes, are uniquely and selectively susceptible to inhibitors of the DNA pol IIIC enzyme. Accordingly, 1,7-dihydro-6H-purin-6-one compounds and the procedures for the synthesis of these compounds, as well as and their use in inhibiting bacterial growth, are disclosed in U.S. Pat. Nos. 6,926,763 and 8,796,292 incorporated by reference herein. For example, ibezapolstat is 2-((3,4-dichlorobenzyl)amino)-7-(2-morpholinoethyl)-1,7-dihydro-6H-purin-6-one. Ibezapolstat, for example, displays antibacterial activity directed against a spectrum of Gram-positive bacteria and has proven useful in the treatment of C. difficile infections. In general, DNA pol IIIC inhibitor mechanism of action targets low G+C (fewer G and C DNA bases than A and T bases) content Gram-positive bacteria (e.g. Firmicutes and Bacillales). The DNA polymerase IIIC (DNA pol IIIC) enzyme is essential for replication in low G+C content Gram-positive pathobionts (microorganisms with genomes comprising fewer guanine and cytosine bases than adenine and thymine/uracil bases). Therefore, an LDN analog targeting the DNA pol IIIC is selective or may be considered to selectively target Gram-positive pathobionts having low G+C genome content. At the same time, such an LDN analog may be inactive against other host microbiota such as Actinobacteria or Bacteroidetes. In other words, Gram-positive bacteria comprising low G+C genome content may be more susceptible to LDN analog treatment whereas other bacteria (beneficial Firmicutes, Actinobacteria, Bacteroidetes and/or Gram-negative bacteria) may not be susceptible to LDN analog treatment. Although the majority of Gram-positive bacteria require the DNA pol IIIC enzyme to replicate, DNA pol IIIC inhibitor susceptibility varies among strains. In addition, it was found that susceptibility to a small-molecule DNA pol IIIC inhibitor (for example, an LDN analog) differed among the strains of beneficial Firmicutes tested. Surprisingly, not only did susceptibility differ among the heterogenous group of different species but also among isolates belonging to the same species (Table 4). As seen in Table 1, in comparison with references strains of Clostridium butyricum, one SNP was identified between a susceptible strain C. butyricum 1008 Y240D (Tyr240Asp) and two SNPs were identified between the less susceptible strain C. butyricum 1007, Y38D and D146E (Tyr38Asp and Asp146Glu). These results suggest that small molecule inhibitors of DNA pol IIIC may be designed to target these aspects and provide enhanced selectivity toward low G+C Gram-positive pathobionts. Further, inhibitors such as LDN analogs provided may avoid antibiotic resistance.

The present disclosure may provide methods of improving the health of the gut microbiome. The methods and compositions provided herein may simultaneously reduce populations of physiologically harmful low G+C content Gram-positive pathobionts in the gut microbiome of a subject while also promoting the persistence, enhanced growth, and/or regrowth of physiologically beneficial gut microbiota.

In some aspects, the LDN analog disclosed may have an influence on flagellar genes, such as fliA, flgB, fliC-VIP, of some bacteria which may result in reduced motility of these organisms. The bacteria may be gram-positive, and may include but is not limited to C. difficile.

Several bacterial species have a flagellum and some species have multiple flagellum, i.e. flagella. Although flagella may be considered a motility organelle, enabling movement and chemotaxis, flagella may exhibit other functions that differ between bacterial species and vary during bacterial life cycle. Further, bacterial motility may play a role important in bacterial colonization and survival. For instance, flagella may allow bacteria to swim and swarm, and therefore give such bacterial species an advantage in terms of accessing nutrients, avoiding predators, and colonizing new environments. In addition, flagella have been shown to participate in protein export, host cell adhesion, cell invasion, auto-agglutination, colonization, the regulation of biofilm formation, and may also be implicated in secretion of non-flagellar bacterial proteins that may be involved in the virulence process. Different bacterial species employ different mechanisms for motility, including twitching, swimming, gliding, and twirling. In addition to flagella, motility may also involve the use of pili and other appendages, which may be affected by environmental factors as well as the physical and chemical properties of a surface. For example, some bacterial species may use motility to communicate and form biofilms (Harshey R. M. (2003). Bacterial motility on a surface: many ways to a common goal. Annual review of microbiology, 57, 249-273). Consequently, flagella may strongly influence the virulence of a bacterial pathogen and may thus play an important role in bacterial pathogenesis.

Some examples of gram-positive bacterial species having at least a single flagellum include Listeria monocytogenes, Clostridium botulinum, Clostridium tyrobutyricum, Staphylococcus aureus, Bacillus subtilis, and C. difficile.

Bacterial flagella may be positioned in one location and provide forward movement (polar) or several flagella may be spread over the surface (peritrichous) of the bacterium to allow for tumbling in place. A flagellum may be a long, whip-like appendage that protrudes from the surface of motile bacteria and rotates to propel the organism. A flagellum includes three major parts: a filament, which acts as a propeller for bacterial motility and contains the protein flagellin (Flg), a basal body, which contains proteins involved in filament rotation, and a hook, which transmits motor torque to the filament. The hook attaches the filament to the basal body and is made up of a hook protein, FlgE. The filament may be a long helical structure composed of flagellin, FlgA, and is attached to the hook protein. The basal body is the motor that powers the rotation of the filament and it is composed of several proteins. including FliG, FliF, and FliM and the flagellar basal-body rod may include FlgB and FlgC. In addition, flagellar chaperone protein, FlgA may play a critical role in early flagellar development (Duan, Q., et al., Flagella and bacterial pathogenicity. Journal of basic microbiology, 53(1), 1-8 (2013).). Regulation of C. difficile motility, for example, may occur at least in part at the level of flagellar gene expression.

Among other virulence factors, C. difficile express a peritrichous flagella as well as toxins, which together, promote diarrheal disease symptoms, pathology, and inflammation. Specifically, C. difficile flagella may promote bacterial motility and adherence to intestinal tissue which in turn aids in the colonization of the intestine that is a necessary prerequisite to diarrheal disease symptoms. Further, it has been observed that the level of adherence of flagellated strains to the mouse cecum is 10-fold higher than the level of adherence of non-flagellated strains (Tasteyre, A., et al., Role of FliC and FliD flagellar proteins of Clostridium difficile in adherence and gut colonization. Infect. Immun. 69:7937-7940 (2001).). It has also been suggested that flagellar function and regulation may contribute to differences in transmissibility and virulence observed between different C. difficile strains. (Twine S M et al. Motility and flagellar glycosylation in Clostridium difficile. J. Bacteriol. 191:7050-7062 (2009).) Recent studies also reveal that C. difficile, and possibly other gram-positive anaerobic bacteria, may have the ability to phase vary (i.e. switch between different phenotypes in response to environmental changes) its virulence factors to survive in different environments. Thus, the pathogenicity of C. difficile, including the development of antibiotic-resistance, may be one result of phase variation and such phase variation may be influenced by the expression of flagellar genes (Anjuwon-Foster, B. R., & Tamayo, R., Phase variation of Clostridium difficile virulence factors, Gut Microbes, 9:1, 76-83 (2018).).

### Therapeutic Administration of Compounds

In some aspects, the LDN analog provided herein may selectively inhibit the growth of harmful Firmicutes such as C. difficile. Further, the compositions and methods provided may inhibit the growth of Bacillales including members of the Staphylococcaceae family such as S. aureus. Thus, the methods and compositions described in the present disclosure may promote a healthy and balanced gut microbiome while also reducing or eliminating harmful pathobionts which may include MDR strains of low G+C Gram-positive pathobionts such as C. difficile, S. aureus, drug resistant S. pneumoniae and/or Enterococcus faecium. In addition, the LDN analogs provided may also effectively treat other infections which may include nosocomial infections. In some aspects, the compositions and methods described herein may reduce the likelihood of or prevent the recurrence of infections or dysbiosis caused by low G+C Gram-positive pathobionts, including C. difficile infection.

In addition, the methods and compositions provided in this disclosure may improve the health of a gut microbiome via the administration of an effective amount of the LDN analog. The subject need not be suffering from dysbiosis or an infection. The proportions of phyla of bacteria in the subject's gut microbiome may be adjusted to a healthier balance compared to the person's gut microbiome prior to the administration of the LDN analog. For example, the percentage of Actinobacteria may increase by around about 5-50% upon exposure to a LDN analog.

In some aspects, in patients having a C. difficile infection, or dysbiosis, the LDN analog causes an overgrowth of healthy gut microbiota such as Actinobacteria and Firmicute phyla species, and an increased proportion of healthy microbiota, such as Clostridiales order taxa, during and after treatment. In further embodiments, in patients having a C. difficile infection, or dysbiosis, the LDN analog initially increases Actinobacteria abundance, followed by decreased abundance of Bacteroidetes and an increased abundance of Lachnospiraceae and Ruminococcaceae, within 2-3 days of the commencement of treatment. In still further embodiments, an overgrowth of healthy gut microbiota accompanying treatment with the LDN analogue results in treatment a 100% clinical cure at day 12 and 100% sustained clinical cure at day 38. For example, CDI may be completely eliminated with no recurrences of infection and with an acceptable adverse event profile.

In some embodiments, treatment with the LDN analogue may attenuate bacterial infection by targeting one or more virulence factors. In some embodiments, virulence factors may be selectively targeted such that deleterious pathobionts in the gut microbiome are reduced without affecting the healthy microbiota. For example, the LDN analogue may inhibit flagellar gene expression in some Gram-positive organisms including but not limited to C. difficile. In some examples, treatment of CDI with the LDN analogue may decrease total toxin A and/or B production. By targeting bacterial virulence factors, the LDN analogue may reduce overall bacterial fitness. For example, the LDN analogue may modulate bacterial morphology and impair cell division. In some embodiments, the LDN analogue may attenuate or eliminate CDI by inhibiting biofilm formation. For example, the LDN analogue may reduce biomass of biofilms embedded with or comprising C. difficile.

In some aspects, in patients not experiencing a C. difficile infection or dysbiosis, the LDN analog causes an increased abundance of Actinobacteria, primarily Bifidobacteriales or Coriobacteriales, during or after dosing. In patients with or without a C. difficile infection or dysbiosis, the LDN analog preserved the proportion of Lachnospiraceae family and the abundance of Clostridiales family. In some aspects, the LDN analog improves the health of a gut microbiome by improving intestinal homeostasis by increasing the amount of C. coccoides. In some further aspects, the LDN analog may stimulate the immune system to reduce inflammation and allergic diseases and cellular components and metabolites, like butyrate, secondary bile acids and indolepropionic acid. For example, the LDN analogue may support the growth of C. coccoides in the gut microbiome thereby allowing C. coccoides to producing short-chain fatty acids (SCFAs) which in turn may inhibit the production of pro-inflammatory cytokines.

Alternatively, or in addition, the LDN analogue may support the growth of bacterial species that produce anti-inflammatory cytokines. In some further embodiments, the LDN analog is used as a probiotic to energize intestinal epithelial cells and strengthening the intestinal barrier. In further aspects, the LDN analog is used prophylactically to prevent, minimize, or reduce dysbiosis.

LDN analogs may fulfill essential criteria for an ideal antibiotic for use against antibiotic resistant bacterial pathogens responsible for many nosocomial infections. For example, the DNA pol IIIC inhibitor ibezapolstat achieves high colonic concentrations with minimal systemic absorption; has potent activity against C. difficile while causing minimal disruption of the gut microbiome; and is well tolerated in healthy volunteers and CDI patients. Alternatively, or in addition, LDN analogs may be used as a prophylactic against dysbiosis.

In accordance with the methods of the invention, the LDN analogs provided herein may be administered to a subject or patient in a variety of forms depending on the selected route of administration, as will be understood by those skilled in the art. For human or animal use, LDN analogs may be administered by the oral, buccal, rectal and vaginal routes, or by topical administration, and the pharmaceutical compositions formulated accordingly. Preferably, the LDN analog is administered in an oral dosage form. Without limitation, for oral administration, the composition can be, for example, in the form of tablets, capsules, granules, liquid solutions and suspensions. The composition may also be administered via suppository or enema. For human or animal use, the formulations of this invention can be administered by parenteral administration, for example, intravenous, subcutaneous, intramuscular, intraorbital, ophthalmic, intraventricular, intracranial, intracapsular, intraspinal, intracisternal, or intraperitoneal administration, or by intranasal, aerosol, scarification, oral, buccal, rectal, vaginal, or topical administration. The formulations of this invention may also be administered by the use of surgical implants which release the compounds of the invention, either as a bolus or slowly over a pre-selected period of time.

The LDN analog may be administered to an animal, preferably a human, alone or in combination with pharmaceutically acceptable excipients, as noted above, the proportion of which is determined by the solubility and chemical nature of the compound, chosen route of administration, and standard pharmaceutical practice. The LDN analog may be administered to adults or to children. The dosage of the compounds of the invention, and/or compositions comprising a compound of the invention, can vary depending on many factors, such as the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, the compounds of the invention can be provided in an aqueous physiological buffer solution containing about 0.1 to 10% w/v compound or in a solid dosage form such as a tablet or capsule. General dose ranges are from about 0.01 mg/kg to about 1 g/kg of body weight per day. Oral dosages of ibezapolstat may include amounts from about 10 mg to 1000 mg per day, preferably 100 mg to 900 mg per day, and more preferably at about 150, 300, 600, or 900 mg per day.

The LDN analogs may be formulated into pharmaceutical compositions for administration to human or animal subjects in a biologically compatible form suitable for administration in vivo or in vitro. Accordingly, the present disclosure provides a pharmaceutical composition including a compound of the invention in admixture with an excipient.

The compounds described herein are useful for the treatment of microbial infections of the gut in humans caused by Gram-positive bacteria, including strains resistant to common antibiotic drugs. They are also useful for the treatment of related Gram-positive bacterial infections in animals such as pigs, cows, horses, goats, chickens, turkeys, sheep, rats, mice, and rabbits, and for eliminating or avoiding bacterial or mycoplasma infections of eukaryotic cell cultures or other media, e.g., foods, cosmetics, medical devices, and hospital supplies.

The compounds of the invention can be formulated for pharmaceutical, veterinary, and tissue culture use, optionally together with an acceptable diluent, carrier, or excipient and/or in unit dosage form. In using the compounds of the invention, conventional pharmaceutical, veterinary, or culture practice can be employed to provide suitable formulations or compositions, all of which are encompassed by the pharmaceutical compositions of this invention.

Without limitation, parenteral formulations can be, for example, in the form of liquid solutions or suspensions for oral administration, formulations can be, for example, in the form of tablets, capsules, liquid solutions and suspensions (wherein such solutions and suspensions are particularly for formulations intended for pediatric use); and for intranasal administration, the formulations can be, for example, in the form of powders, nasal drops, or aerosols. Other suitable formulations for parenteral, oral or intranasal delivery of the compounds of this invention will be well known to those of ordinary skill in the art. Methods well known in the art for making formulations can be found in, for example, “Remington's Pharmaceutical Sciences.” formulations for parenteral administration may contain as excipients sterile water or saline, ethanol, propylene glycol, polyalkylene glycols such as polyethylene glycol, oils of vegetable origin, hydrogenated naphthalene's, or biocompatible, biodegradable lactide polymers. Polyoxyethylene-polyoxypropylene copolymers can be used to control the release of the present compounds. Other potentially useful parenteral delivery systems for the pounds of the invention include ethylene-vinyl acetate copolymer particles, osmotic pumps, implantable infusion systems, and liposomes. Formulations for inhalation may contain lactose as an excipient, or can be aqueous solutions containing, for example, polyoxyethylene-9-lauryl ether, glycocholate and deoxycholate, or can be oily solutions for administration in the form of nasal drops, or can be gels to be applied intranasally. Formulations for parenteral administration may also include glycocholate for buccal administration, methoxy-salicylate for rectal administration, or citric acid for vaginal administration.

The concentration of the compound in the formulations of the invention will vary depending on a number of factors, including the dosage to be administered, and the route of administration. In general, the compounds of the invention can be provided in an aqueous physiological buffer solution containing about 0.1 to 10% w/v compound for parenteral administration. General dose ranges are from about 0.01 mg/kg to about 1 g/kg of body weight per day, e.g., from about 0.01 mg/kg to 100 mg/kg of body weight per day. The dosage to be administered depends upon the type and extent of progression of the infection being addressed, the overall health of the patient, and the route of administration. For topical and oral administration, formulations and dosages can be similar to those used for other antibiotic drugs.

In one embodiment, a compound or composition of the invention is administered to an animal, such as a human, patient, that has been diagnosed with a Gram-positive bacterial infection. The compounds can also be administered to the animal or human to inhibit or reduce the likelihood of a Gram-positive bacterial infection, particularly in an animal or human susceptible to such infections (including, without limitation, a human patient who is immunodeficient or immunocompromised or one who has recently undergone a medical procedure). In other embodiments, cultured eukaryotic cells are treated with the new compositions, or the compositions are added to inhibit or reduce the likelihood of such infections (e.g., prophylactic treatment). The compounds of the invention may also be used the prevent bacterial growth in food products, cosmetics, and medical supplies, and on surfaces.

The compounds can be administered both prophylactically and after infection has occurred. Prophylaxis can be most appropriate for immunocompromised human patients and animals and for patients and animals following surgery or dental procedures. This list of relevant conditions for application of the methods of the invention is not intended to be limiting, and any appropriate infection responsive to the compounds can be treated using the methods and/or compounds described herein.

### Reduction of Transmissibility of Infection

In some aspects, the LDN analog provided herein may be used to reduce the transmissibility of C. difficile or other Gram-positive pathogens, for example, by reducing the motility of the bacterial cells. In some embodiments, the LDN analogue may inhibit transmission by targeting one or more virulence factors thus preventing the spread of harmful disease. In some embodiments, the LDN analogue may selectively inhibit virulence factors such as flagellar genes, toxin production, and/or biofilm formation. For example, the LDN analogue may reduce pathogenic transmission by inhibit flagellar gene expression and thus reduce or eliminate bacterial motility. In addition, the LDN analogue may modulate bacterial morphology and impair cell division. In some embodiments, surface treatment with the LDN analogue reduce or inhibit biofilm formation. In some examples, the LDN analogue may reduce biomass of biofilms embedded with or comprising C. difficile.

The compounds may also be used to treat or coat media or surfaces to prevent or reduce the extent of microbial growth. For example, the compounds of the invention can be mixed with eukaryotic culture media (e.g., solid or liquid media) in order to prevent Gram-positive bacterial growth. In addition, the compounds of the invention may be used in disinfectant formulations for treating surfaces, e.g., a liquid formulation for cleaning and disinfecting surfaces, such as those in kitchens, bathrooms, hospitals, or other areas of medical treatment or potential microbial growth. Medical devices and other surfaces can also be treated or coated with compounds of the invention in order to control microbial growth. Medical devices include those that are wholly or partially implanted in an animal and those external to an animal. Examples of medical devices include, without limitation, catheters, dialysis pumps, blood collection equipment, stens, and drug delivery devices. Standard formulations for the use of the compounds of the invention for surface treatments or in coatings are known to those skilled in the art.

### EXAMPLES

The foregoing description and examples have been set forth merely to illustrate the invention and are not meant to be limiting. Since modifications of the described embodiments incorporating the spirit and the substance of the invention may occur to persons skilled in the art, the invention should be construed broadly to include all variations within the scope of the claims and equivalents thereof.

### Example 1

### DNA Polymerase IIIC Production

The DNA polymerase IIIC coding genes may be amplified from genomic DNA. The primers may be designed so the 5′-terminal and 3′-terminal contain BamHI and XhoI cutting site respectively. The fragment may be directly inserted into expression plasmid pET-28a (+) between BamHI and XhoI sites. The recombinant protein may have full length of original protein and a His tag, a thrombin site and a T7 tag at N-terminus. The coding sequence of recombinant protein may be confirmed by Sanger sequencing using 8 primers to cover the entire coding regions.

The plasmid was transferred into BL21 (DE3) competent E. coli cells. The positive colonies may be inoculated into 1L LB medium and incubated at 37° C. with 200 rpm rotation to reach OD600-0.6. The inducer IPTG 1 mM may be then added to induce the expression for 18 hours at 16° C. The cells may be harvested and suspended in lysis buffer (25 mM Tris-HCl pH=7.5, 0.15 M NaCl, 20 mM imidazole, 2 mM β-mecaptoethanol and 1×Roche proteinase inhibitor cocktail). The cells may be lysed by sonication, and the debris may be spun down at 50000 g for 1 hour.

The protein may be isolated first by passing through the lysate through Ni column. The column may be washed with 50 column volume (CV) binding buffer (lysis buffer without proteinase inhibitor) and then 10 CVs of washing buffer (binding buffer with total 40 mM imidazole). The protein may be eluted out with elusion buffer (binding buffer with total 300 mM imidazole).

The crude extract may be then further purified by size exclusion chromatography using a Superdex 200 increase 10/300 column with 25 mM Tris-HCl pH 7.5, 0.15 M NaCl, 5% Glycerol and 1 mM DTT. The final product was stored as 50-100 μl aliquots at −80° C.

FIG. 1 shows changes in proportional abundance of relevant gut microbiome species over time in healthy volunteers or patients with CDI given an inhibitor of DNA pol IIIC. Using primers targeted to Clostridium cluster XIVa (C. coccoides) and Clostridium cluster IV (C. leptum) the proportion of these relevant Firmicutes were quantified over time using qPCR. As seen in FIG. 1A, samples taken from a Phase 1 study of healthy volunteers over the course of 12 days showed that treatment with 450 mg of a small molecule DNA pol IIIC inhibitor twice a day increased the amount of C. coccoides. Clostridium cluster XIVa, also known as Clostridium coccoides group, consists of 21 species. These commensal bacteria may play an important role in intestinal homeostasis. In addition, members of the XIVa cluster have been shown to attenuate inflammation and allergic diseases and cellular components and metabolites, like butyrate, secondary bile acids and indolepropionic acid, of these species may play a probiotic role in the intestine primarily through energizing intestinal epithelial cells, strengthening intestinal barrier and interacting with the immune system.

As shown in FIG. 1B, participants suffering from CDI made up the cohort in Phase 2a of the study. In contrast to the healthy volunteers in Phase 1 of the study, subjects in Phase 2a exhibited low amounts of both Clostridium cluster XIVa and cluster IV throughout the study. Table 2 and table 3, however, show that Actinobacteria increased in abundance after starting the DNA pol IIIC inhibitor (primarily Bifidobacteriales or Coriobacteriales) and persisted for the entire dosing period. In comparison to the phase 1 study, the phase 2a CDI study baseline microbiota had a lower proportion of Actinobacteria and Firmicutes and increased Bacteroidetes. In CDI patients, Actinobacteria increased in abundance (primarily Coriobacteriales) followed within 2-3 days by decreased abundance of Bacteroidetes, and an increased abundance of Lachnospiraceae and Ruminococcaceae. In addition, the Phase 1 and Phase 2a studies both show that the proportion of Lachnospiraceae Family is preserved and Clostridiales Family abundance was also preserved.

### Broth Microdilution Based Minimum Inhibition Concentration (MIC) Determination

On the day of testing, the compounds may be dissolved in pure DMSO (Sigma 276855-2L) to 20 mM as stock. In a v-bottom 96-well plate (Axygen-wipp 2280), 30 μl DMSO was added into well 1 to well 12 by manual pipetting. Into well 1, 30 μl of compound DMSO stock (20 mM) may be added and mixed by pipetting. Two-fold serial dilutions may be performed by transferring and mixing 30 μl of solution from well 1 to well 2, then well 2 to well 3 and so forth until well 11. The well 12 may be loaded with 30 μl DMSO without compounds. This may be drug ‘mother plate’. From well 1 to well 12, the drug concentrations in the mother plate may be 10, 5, 2.5, 1.25, 0.625, 0.3125, 0.156, 0.078, 0.039, 0.02, 0.01 and 0 mM in DMSO. A multi-pipette may be used to perform the serial dilutions. The concentration may be adjusted according to the potency of the compounds. An Echo® Acoustic liquid handling system may be used to replace manual pipetting to make the daughter plates.

One day prior to the day of MIC testing, bacterial strains may be streaked out from −80° C. glycerol stocks onto MHA plates and incubated at 37° C. for 20 hours. Streptococcus pneumoniae may be streaked on blood agar and incubated at 37° C. in 5% CO₂. The single colonies were picked by using an inoculation loop (Greiner-731175) and suspended into 5 ml sterile saline. The turbidity of the suspension may be adjusted to 0.10 (Siemens MicroScan turbidity meter), equal to ˜1.0×10⁸ cfu/ml. The bacterial suspension may be diluted 100× in corresponding test medium (Table 1.). This may be used to inoculate daughter plates.

For preparing u-bottom 96-well ‘daughter plates’ (Costar 3788), 98 μl of test medium was added into each well of a daughter plate. An aliquot of 2 μl solution from mother plate may be then replicating transferred into the daughter plate using a multi-pipette.

An aliquot of 100 μl of the bacterial suspension may be inoculated into each well of the daughter plates using a multi-pipette. Each well contained ˜5.0×10⁵ cfu/ml bacteria, 1% DMSO and serially diluted compounds at 100, 50, 25, 12.5, 6.25, 3.125, 1.56, 0.78, 0.39, 0.2, 0.1 and 0 μM from well 1 to well 12 respectively, in 200 μl corresponding test medium.

The plates may be incubated in ambient atmosphere and in a 37° C. incubator for 20 hours.

The MIC values were determined by visual inspection as the lowest compound concentration that completely or significantly inhibits the growth of bacteria in the test medium.

Compounds of the present invention were tested for antibacterial activity against a variety of bacterial organism including Bacillus subtilis, Staphylococcus aureus, Enterococcus faecalis, Enterococcus faecium, Streptococcus pneumoniae and Escherichia coli. Compounds described in Examples 1, 18, 19, 28, 30, 32 and 34 had Ki of 0.31-1.45 μM against Bacillus subtilis DNA pol IIIC enzyme and MIC of 0.25-4.0 μg/ml against strains of Gram-positive organisms. The compounds showed weak Gram-negative activity with MICs of 16->64 μg/ml against Escherichia coli.

FIG. 2 shows the results of MIC determinations performed on select Firmicutes isolated from samples taken during Phase 2a of the study. It was found that susceptibility to the DNA pol IIIC inhibitor differed among the strains of beneficial Firmicutes tested. Surprisingly, not only did susceptibility differ among the heterogenous group of different species but also among isolates belonging to the same species (Table 4). Isolated of Clostridium butyricum strains (C. butyricum 1008 and C. butyricum 1007) were sequenced and compared. As seen in FIG. 2A, C. butyricum 1008 (1.5 ug/mL) exhibits a much lower susceptibility than C. butyricum 1007. In contrast to C. butyricum 1008, C. butyricum 1007 exhibited attenuated susceptibility (C. butyricum 1007>100 ug/mL). FIG. 2B is a rendering of the molecule structure of the two C. butyricum strains. As seen in Table 1 below, in comparison with references strains of Clostridium butyricum, one SNP was identified in the susceptible strain C. butyricum 1008 Y240D (Tyr240Asp) and two SNPs were identified in the less susceptible strain C. butyricum 1007, Y38D and D146E (Tyr38Asp and Asp146Glu). These results suggest that small molecule inhibitors of DNA pol IIIC provide enhanced selectivity toward low G+C Gram-positive pathobionts. Further, inhibitors such as LDN analogs provided may avoid antibiotic resistance towards targeted pathobionts.

TABLE 1

| | nucleotide | nucleotide | | protein | |
|---|---|---|---|---|---|
| Sample | position | change | protein | change | COMMENTS |
| APT_1008 | 163003 | A > C | WP 071982014.1 | Tyr240Asp | OM YfiO superfamily |
| APT_1007 | 904344 | T > G | WP 071982178.1 | Tyr38Asp | N-acetylmuramoyl-L- |
| | | | | | alanine amidase family |
| | | | | | protein |
| APT_1007 | 2512780 | A > T | WP 071982697.1 | Asp146Glu | serine protease |

In addition, results suggest that certain beneficial Firmicutes or commensals exhibited varying susceptibility to the small molecule inhibitor of DNA pol IIIC. Using isolated gut microbiota species, the DNA pol IIIC inhibitor was inactive (MIC>64 μg/mL) against representative Actinobacteria (Bifidobacteriaceae and Coriobacteriaceae) and certain Firmicutes (Lachnospiraceae and Lactobacillusceae) but highly active against strains of C. difficile (MIC≤2 μg/mL).

### Example 2

### Microbiome Studies for the Phase I Healthy Volunteer Study:

Background: The microbiome of the healthy gut is composed of two major bacterial groups called phyla. Firmicutes (Gram-positive spore forming organisms) and Bacteroidetes (Gram-negative non-spore forming organism) are most common. A third phylum, Proteobacteria (Gram-negative facultative anaerobes), is present in low abundance but generally comprise 2-5% of the healthy microbiome. A fourth phylum, Actinobacteria (mostly gram-positive, majority saprophytes) is present in large proportion in children and may generally decrease in overall proportion with age. Patients suffering from C. difficile infection are in a state of dysbiosis, and often have an increased proportion of Proteobacteria, for example, an overabundance of Proteobacteria or “Proteobacteria bloom,” and a reduced number of Firmicutes and Bacteroidetes.

Ibezapolstat studies: Using stool samples from the phase 1 healthy volunteer study and shotgun metagenomic sequencing, it was demonstrated that treatment with ibezapolstat over 10 days resulted in a significantly different microbiome profile in subjects compared to subjects receiving placebo. The difference was a larger proportion of Actinobacteria and Firmicute Phylum in ibezapolstat treated subjects vs. a larger proportion of Proteobacteria in vancomycin treated subjects.

### Methods and Materials

### Description of Clinical Trial:

Six healthy volunteers were given 450 mg ibezapolstat twice a day for 10 consecutive days. Stool samples were collected daily (Garey K W, et al. A randomized, double-blind, placebo-controlled, single and multiple ascending dose Phase 1 study to determine the safety, pharmacokinetics and food and faecal microbiome effects of ibezapolstat administered orally to healthy subjects. J Antimicrob Chemother 2020; 75(12):3635-3643). Institutional review board approval was obtained (Midlands Institutional Review Board IRB #222220170383) and all volunteers signed an informed consent form prior to performing any study procedures. For this analysis, stool samples were collected daily for days 0 (baseline)-13 and day 30 follow-up if available from subjects given ibezapolstat 450 mg twice daily. Stool samples were immediately frozen at −80C prior to shipping to the University of Houston on dry ice for analysis.

### Stool DNA Extraction and Shotgun Metagenomic Sequencing:

Stool DNA was extracted using a DNAeasy Power Soil Pro kit (Qiagen, catalog number 1288-100) in a QiaCube automated DNA extraction system as previously described. (Garey K W, et al. A randomized, double-blind, placebo-controlled, single and multiple ascending dose Phase 1 study to determine the safety, pharmacokinetics and food and faecal microbiome effects of ibezapolstat administered orally to healthy subjects. J Antimicrob Chemother 2020; 75 (12): 3635-3643) Shotgun metagenomic sequencing was carried out at the University of Houston Sequencing and Gene Editing Core (Houston, TX USA) using the Nextera DNA Flex Library Prep Kit for DNA library preparation and an Illumina NextSeq 500 platform for sequencing. CLC Genomic Workbench version 12 (Qiagen) was used for metagenomic assembly and creation of the abundance table.

16S Ribosomal RNA (rRNA) Gene Sequencing:

16S rRNA sequencing was performed to characterize microbial taxonomy as described in Gonzales-Luna 2021. The V3-V4 region of the 16S rRNA gene was sequenced using an Illumina-based sequencing platform with a minimum of 15,000 reads per sample to assess the gut microbiome community structure. Quality filtered sequence reads with at least 97% similarity were clustered into Operational Taxonomic Units (OTUs) and representative sequences from each OTU were assigned a taxonomic identity at the species level by searching against the NCBI 16S rRNA sequence database (release date Sep. 1, 2018) using NCBI BLAST+ package v2.8.1 2018.

### Shotgun Metagenomic Sequencing:

The DNA extracted from fecal samples previously used for 16S rRNA sequencing was shotgun metagenome sequenced using an Illumina-based platform for analysis of microbiome functional genes. The functional gene profiling of the shotgun metagenome was performed using HUMAnN2 v0.11.2 pipeline.35 In the preprocessing step, quality filtering of sequencing reads was performed and followed by screening and removal of contaminant host (human) reads. Trimmomatic v0.38 was used for the filtering and trimming of raw sequence data with the default cut-off settings. The reads were searched against a human genome database in paired-end mode using bowtie2 algorithm and were discarded if they were mapped to the database. To obtain the gene family profiles, these quality-controlled metagenome sequences were first searched against a nucleotide database (ChocoPhlAn) using bowtie2 and then against a protein database (UniRef90) using diamond. All the identified gene families were annotated using UniRef90 and pathways using MetaCyc identifiers.

### Microbiome Analysis

Using the sequencing described above, the changes in proportional abundance of gut microbiome species over time in healthy volunteers receiving ibezapolstat was analyzed. Metagenomic data showing the proportion of isolated Firmicutes per day during the study are shown in Table 2 below. Baseline gut microbiota from healthy volunteers were primarily Firmicutes, Bacteroidetes, or Actinobacteria. Actinobacteria increased in abundance after starting IBZ (primarily Bifidobacteriales or Coriobacteriales) and persisted for the entire dosing period. As shown in Table 2 below, the most commonly isolated Genus (species) during the Phase 1 study of healthy volunteers was Streptococcaceae (Lactococcus lactis and Streptococcus thermophilus); Lachnospiraceae (Blautia, Roseburia); and Ruminococcaceae (Faecalibacterium, Ruminococcus).

TABLE 2

| | Healthy Volunteers | | | | | |
|---|---|---|---|---|---|---|
| Family | Baseline | 3 | 5 | 8 | 10 | 12 |
| Erysipelotrichaceae | 2% | 4% | 7% | 7% | 18% | 25% |
| Eubacteriales Family XIII | | | | | | |
| Lachnospiraceae | 26% | 23% | 21% | 16% | 24% | 47% |
| Lactobacilloae | | | | 12% | 5% | 6% |
| Peptostreptococcaceae | | | | | | |
| Ruminococcaceae | 68% | 37% | 20% | 7% | 3% | 4% |
| Streptococcaceae | | 27% | 43% | 49% | 41% | 14% |
| Veillonellaceae | | 3% | | | | |
| Other | 4% | 6% | 9% | 9% | 9% | 5% |

### Example 3

Microbiome Data from Phase 2a Clinical Trial of Ibezapolstat for CDI
Phase 2 clinical trials:

The Phase 2 clinical trial was designed to evaluate ibezapolstat in the treatment of CDI.

Phase 2a of this trial was an open label cohort of 10 subjects from study centers in the United States. In this cohort, 10 patients with diarrhea caused by mild or moderate C. difficile diagnosed via toxin EIA+ were treated with ibezapolstat 450 mg orally, twice daily for 10 days. All patients were followed for recurrence for 28±2 days. Stool was collected during course of therapy and at follow up. Patient fecal samples were evaluated for C. difficile culture and microbiome changes. The study demonstrated 100% clinical cure at day 12 and 100% sustained clinical cure at day 38. Favorable microbiome changes included overgrowth of Actinobacteria and Firmicutes phylum species while on therapy. These findings support that microbiome effects may be predictive of beneficial patient outcomes including low rates of recurrence. The infection was eliminated 100% with no recurrences of infection (100%), and with an acceptable adverse event profile.

### Methods and Materials

### Safety Assessments

Safety evaluations included AE assessment, physical examination, vital signs, clinical laboratory tests (chemistry, hematology, and urinalysis), and electrocardiogramaty endpoints for all subjects were recorded including nature, frequency, and severity of AEs. AEs were assessed at each visit beginning from the time of enrollment and classified according to the Medical Dictionary for Regulatory Activities (MedDRA version 15.0). AE severity (mild, moderate, or severe) and causality (unrelated, possibly related, or probably related to the study medication) were assessed by the investigator at each site.

### Microbiology

Stool samples were cultured for C. difficile growth on a selective cycloserine-cefoxitin fructose agar (CCFA) at 37° C. under anaerobic conditions for 48 hours. (Gonzales-Luna A J, Carlson T J. Dotson K M, et al. PCR ribotypes of Clostridioides difficile across Texas from 2011 to 2018 including emergence of ribotype 255. Emerg Microbes Infect 2020; 9(1): 341-7). Isolates were determined to be C. difficile based on growth and morphology and confirmed by PCR for C. difficile toxin and tpi genes. C. difficile was strain typed using a PCR-based ribotyping method as previously described (Gonzales-Luna A J, Carlson T J, Dotson K M, et al. PCR ribotypes of Clostridioides difficile across Texas from 2011 to 2018 including emergence of ribotype 255. Emerg Microbes Infect 2020; 9(1):341-7). Minimum inhibitory concentrations (MICs) were determined for ibezapolstat by broth microdilution in 0.1% sodium taurocholate Brain Heart Infusion (BHI) media (Begum K, Basseres E, Miranda J. et al. In Vitro Activity of Omadacycline, a New Tetracycline Analog, and Comparators against Clostridioides difficile. Antimicrob Agents Chemother 2020; 64(8)).

### Statistical Analysis

An intent-to-treat analysis of patients receiving at least one dose of ibezapolstat was conducted. Descriptive statistics were calculated for efficacy, safety/tolerability, and PK data generated using SAS version 9.4 software (SAS Institute, Inc Cary, NC, USA). Microbiome summary plots and data visualization was prepared using R software version 4.1.1 (R Core Team 2021, Vienna, Austria). (R Core Team (2013). R: A language and environment for statistical computing. R Foundation for Statistical Computing, Vienna, Austria. Proportional changes of bacterial taxa over the 10-day dosing interval were calculated using linear regression models for taxa with at least a one percent proportional change during the study time period. A p-value <0.05 was considered significant.

### Results of Phase 2a Clinical Trial:

Completed Phase 2a CDI clinical trial showed a 100% success rate with favourable microbiome changes. The phase 2a data demonstrated complete eradication of colonic C. difficile by day three of treatment with ibezapolstat as well as the observed overgrowth of healthy gut microbiota, Actinobacteria and Firmicute phyla species, during and after therapy. Additionally, the data demonstrated an increased proportion of healthy microbiota including Clostridiales order taxa. The phase 2a CDI study baseline microbiota had a lower proportion of Actinobacteria and Firmicutes and increased Bacteroidetes. In CDI patients, Actinobacteria increased in abundance after starting IBZ (primarily Coriobacteriales) followed within 2-3 days by decreased abundance of Bacteroidetes, and an increased abundance of Lachnospiraceae and Ruminococcaceae.

### Patients

Ten patients aged 27-75 (±15) years (50% female; 100% white race; 80% Hispanic or Latino ethnicity) were enrolled. All ten patients received ibezapolstat and completed the study. Median number of unformed bowel movements in the 24 hours prior to start of therapy was 4 (range: 3-10). Two of 10 patients received <24 hours of antibiotics, either metronidazole or vancomycin, prior to starting ibezapolstat. No patients were hospitalized prior to or following enrollment.

### Microbiology Results

Metagenomic data from the Phase 2a study showing the proportion of isolated firmicutes per day during the study is shown in Table 3. In CDI patients, Actinobacteria increased in abundance after starting IBZ (primarily Coriobacteriales) followed within 2-3 days by decreased abundance of Bacteroidetes, and an increased abundance of Lachnospiraceae and Ruminococcaceae. The Most commonly isolated Genus (species) during study included Streptococcaceae (Streptococcus salivarus and Streptococcus thermophilus); Lachnospiraceae (Blautia, Roseburia); Ruminococcaceae (Faecalibacterium, Ruminococcus).

TABLE 3

| | CDI Patients | | | | | |
|---|---|---|---|---|---|---|
| Family | Baseline | 3 | 5 | 8 | 10 | 12 |
| Erysipelotrichaceae | 18% | 24% | 10% | 9% | 17% | 6% |
| Eubacteriales Family XIII | | 2% | 1% | 3% | 5% | 4% |
| Lachnospiraceae | 30% | 37% | 38% | 33% | 35% | 44% |
| Lactobacilluseae | 6% | | | | | |
| Peptostreptococcaceae | 2% | 1% | | 5% | 4% | 6% |
| Ruminococcaceae | 7% | 25% | 38% | 30% | 26% | 24% |
| Streptococcaceae | 13% | 3% | 1% | 4% | 5% | 2% |
| Veillonellaceae | 19% | | | 5% | | |
| Other | 5% | 9% | 10% | 9% | 8% | 10% |

### Example 4

### Gram-Positive Selective Spectrum of Ibezapolstat

The purpose of this study was to assess the selectivity of Ibezapolstat against Gram-positive gut microbiota. In vitro and human studies have shown potent activity of Ibezapolstat against C. difficile but selective activity against other beneficial Gram-positive gut microbiota shown to reduce the risk of recurrent CDI.

### Methods and Materials

### Firmicute Stool Isolation

Using stool samples and microbiome data from the Phase 1 and Phase 2a studies outlined above, changes in proportional abundance of gut microbiome species were analyzed over time in healthy volunteers and patients with CDI given Ibezapolstat. In Phase 1, six healthy volunteers were given 450 mg of ibezapolstat twice daily for 10 days. For Phase 1, stool samples were collected daily for days 0 (baseline)-13 and day 30 follow-up if available from subjects given ibezapolstat 450 mg twice daily. Stool samples were immediately frozen at −80C prior to shipping to the University of Houston on dry ice for analysis.

Phase 2a of this trial was an open label cohort of 10 subjects from study centers in the United States. In this cohort, 10 patients with diarrhea caused by mild or moderate C. difficile diagnosed via toxin EIA+ were treated with ibezapolstat 450 mg orally, twice daily for 10 days. Stool was collected during the course of therapy and at follow up.

### Stool DNA Extraction and Shotgun Metagenomic Sequencing:

Stool DNA was extracted using a DNAeasy Power Soil Pro kit (Qiagen, catalog number 1288-100) in a QiaCube automated DNA extraction system as previously described. (Garey K W, et al. A randomized, double-blind, placebo-controlled, single and multiple ascending dose Phase 1 study to determine the safety, pharmacokinetics and food and faecal microbiome effects of ibezapolstat administered orally to healthy subjects. J Antimicrob Chemother 2020; 75 (12): 3635-3643). Shotgun metagenomic sequencing was carried out at the University of Houston Sequencing and Gene Editing Core (Houston, TX USA) using the Nextera DNA Flex Library Prep Kit for DNA library preparation and an Illumina NextSeq 500 platform for sequencing. CLC Genomic Workbench version 12 (Qiagen) was used for metagenomic assembly and creation of the abundance table.

16S Ribosomal RNA (rRNA) Gene Sequencing:

16S rRNA sequencing was performed to characterize microbial taxonomy as described in Gonzales-Luna 2021. The V3-V4 region of the 16S rRNA gene was sequenced using an Illumina-based sequencing platform with a minimum of 15,000 reads per sample to assess the gut microbiome community structure. Quality filtered sequence reads with at least 97% similarity were clustered into Operational Taxonomic Units (OTUs) and representative sequences from each OTU were assigned a taxonomic identity at the species level by searching against the NCBI 16S rRNA sequence database (release date Sep. 1, 2018) using NCBI BLAST+ package v2.8.1 2018.

### Shotgun Metagenomic Sequencing:

The DNA extracted from fecal samples previously used for 16S rRNA sequencing was shotgun metagenome sequenced using an Illumina-based platform for analysis of microbiome functional genes. The functional gene profiling of the shotgun metagenome was performed using HUMAnN2 v0.11.2 pipeline.35 In the preprocessing step, quality filtering of sequencing reads was performed and followed by screening and removal of contaminant host (human) reads. Trimmomatic v0.38 was used for the filtering and trimming of raw sequence data with the default cut-off settings. The reads were searched against a human genome database in paired-end mode using bowtie2 algorithm and were discarded if they were mapped to the database. To obtain the gene family profiles, these quality-controlled metagenome sequences were first searched against a nucleotide database (ChocoPhlAn) using bowtie2 and then against a protein database (UniRef90) using diamond. All the identified gene families were annotated using UniRef90 and pathways using MetaCyc identifiers.

Quantitative PCR (qPCR) Analysis

Quantity and quality of extracted DNA were assayed with Qubit 4 Fluorometer (Invitrogen). Sample DNA was diluted with PCR grade water to 5 ng/μL. The DNA levels of bacterial groups were assessed using specific PCR primers/conditions.¹¹⁻¹⁴ Using the 7300 Real Time PCR System (Applied Biosystems), qPCR was performed on each sample in triplicate in a final volume of 20 μL containing 25 ng DNA template, primers at 0.5 μM, and QuantiTect SYBR Green Mixes (Qiagen). For Eubacteria a FAM-tagged probe at 0.25 μM and TaqPath ProAmp Master Mixes (Qiagen) was used. Threshold cycle values were converted to copies per ng of DNA using a standard curve. Standards were prepared by performing PCR using species specific primers on appropriate bacterial strains or DNA from normal stool. The PCR products were cloned using Invitrogen TOPO PCR Cloning Kit (Invitrogen), and verified by sequencing at the University of Houston Core Facility. A Basic Local Alignment Search Tool (BLAST) search was performed to identify the closest matching database sequence. A range of 10-fold serially diluted plasmid standard DNA (5×10⁸ to 500 copies) was run on each qPCR plate in triplicate. Standard curve R² values were calculated for standards. Copies per gram of stool were calculated, accounting for initial sample DNA concentrations and stool weights. The change in bacterial levels (Δlog10 copies/gram stool) from entry level to each available successive time-point was determined for each participant and median changes calculated.

### Firmicute Stool Isolation (Phase 2a Samples):

Fecal samples homogenized in reduced PBS (0.1 g stool/ml PBS) and serially diluted and plated directly onto YCFA7 agar supplemented with 0.002 g/ml each of glucose, maltose and cellobiose in large (13.5 cm diameter) petri dishes. Isolated colonies were identified by PCR amplification of the full-length 16S rRNA gene (using 7F (5′-AGAGTTTGATYMTGGCTCAG-3′) forward primer and 1510R (5′-ACGGYTACCTTGTTACGACTT-3′) reverse primer followed by Sanger sequencing. Full-length 16S rRNA gene sequence reads were aligned using CLC Genomics (Qiagen) using the Ribosomal Database Project (RDP) as reference to classify reads to Operational Taxonomic Units (OTUs). The full-length 16S rRNA gene sequence of each species-level OTU was compared to the Ribosomal Database Project (RDP) reference database to assign taxonomic designations to the genus level and a BLAST search performed to identify any candidate novel species.

### Whole Genome Sequencing

Genomic DNA was extracted from at least one representative of each identified species. DNA was sequenced on the Illumina HiSeq platform generating read lengths of 150 bp and these were assembled and annotated for further analysis. For whole-genome SNP analysis, cleaned sequence reads were mapped to the reference genome using CLC Genomics and the RedDog pipeline according to the developer's guidelines (https://github. com/katholt/RedDog). Briefly, Bowtie2 version 2.2.3 was used for mapping and SAMtools version 0.1.19 was used for calling SNPs. Only high-quality SNPs were used for phylogenetic analyses.

### Minimum Inhibitory Concentration (MIC) Determinations:

Strains plated onto blood agar (Hardy Diagnostics) with a 24-hour incubation at 37° C. in an anaerobic environment (Coy vinyl anaerobic chamber). After incubation, 3-5 well-isolated colonies were suspended into 5 mL of BHI broth (Criterion Media). The cultures were incubated in an anaerobic chamber for 24 hours to achieve a 0.5 McFarland Standard. Brucella agar with 5% hemin (5 ug/mL) (Sigma), vitamin K (10 ug/mL), and defibrinated sheep blood (Northeast Lab Services). The supplemented brucella agar was then utilized for two-fold serial dilution to create plates with IBZ concentration of 0.5 to 16 μg/mL plates. Plates were allowed to dry in a sterile location. After drying, the plates were covered with foil to prevent any photo-damage to the hemin and placed in the anaerobic chamber for 1 hour to reduce the media. The broth culture was then spotted onto the plates and covered with foil. Plates were allowed to dry completely before inverting them for incubation for 48 hours. Plates were analyzed and MIC recorded.

### Microbiology Result:

Baseline gut microbiota from healthy volunteers were primarily Firmicutes, Bacteroidetes, or Actinobacteria. Actinobacteria increased in abundance after starting ibezapolstat (primarily Bifidobacteriales or Coriobacteriales) and persisted for the entire dosing period. In both Phase 1 and Phase 2a studies, the proportion of Lachnospiraceae Family and abundance of Clostridiales Family were preserved. In comparison to the Phase 1 study, the Phase 2a CDI study baseline microbiota had a lower proportion of Actinobacteria and Firmicutes and increased Bacteroidetes. In CDI patients, Actinobacteria increased in abundance after starting ibezapolstat (primarily Coriobacteriales) followed within 2-3 days by decreased abundance of Bacteroidetes, and an increased abundance of Lachnospiraceae and Ruminococcaceae.

### Beneficial Firmicutes Show Mixed Ibezapolstat Susceptibility

Using isolated gut microbiota species, Ibezapolstat was inactive (MIC>64 μg/mL) against representative Actinobacteria (Bifidobacteriaceae and Coriobacteriaceae) and certain Firmicutes (Lachnospiraceae and Lactobacilluseae) but highly active against strains of C. difficile (MIC<2 μg/mL). Thus, microbiome changes with ibezapolstat may be dependent on underlying composition of the baseline microbiome. In both Phase 1 and Phase 2a cohorts, however, there was an increased abundance of Actinobacteria after starting therapy. IBZ microbiome data coupled with in vitro MIC determinations demonstrated persistence or regrowth of healthy microbiota associated with beneficial physiologic effects. The results of the MIC determinations of Firmicutes are shown in Table 4. Among the isolated beneficial Firmicutes, ibezapolstat showed mixed susceptibility.

TABLE 4

| Species | N | MIC50 | Min | Max |
|---|---|---|---|---|
| Blautia sp. | 1 | 12.5 | | |
| Clostridium butyricum | 5 | 12.5 | 1.5 | 100 |
| Clostridium subterminale | 1 | 1.0 | | |
| Clostridium tertium | 2 | 12.5 | 3.125 | 12.5 |
| Coprococcus sp | 1 | 25 | | |
| Dorea longicatena | 1 | 3.125 | | |
| Enterococcus avium | 2 | 12.5 | 12.5 | 12.5 |
| Enterococcus durans | 1 | 100 | 100 | 100 |
| Enterococcus faecalis | 5 | 12.5 | 12.5 | 50 |
| Enterococcus mundtii | 1 | 12.5 | | |
| Enterococcus pseudoavium | 1 | 100 | | |
| Erysipelatoclostridium ramosum | 5 | 6.25 | 3.125 | 100 |
| Flavonifractor plautii | 1 | 25 | | |
| Lachnoclostridium pacaense | 1 | 25 | | |
| Longibaculum sp. | 1 | 50 | | |
| Melissococcus plutonius | 1 | 6.25 | | |
| Paeniclostridium sordellii | 4 | 1.5 | 0.75 | 12.5 |
| Clostridioides difficile | 6 | 0.5 | 0.25 | 1 |

Although Firmicutes generally possess DNA Pol IIIC enzymes, some members of the Firmicute Phyla (also referred to as Bacillota phylum) were more susceptible to ibezapolstat. Among the heterogeneous population of Firmicutes tested, different strains of Clostridium butyricum showed differing susceptibility. As seen in FIG. 1A, C. butyricum 1008 was more susceptible to ibezapolstat (IBZ MIC: 1.5 ug/mL) when compared to C. butyricum 1007 (IBZ MIC: >100 μg/mL). Following whole genome sequencing as previously described, three single nucleotide polymorphisms (SNPs) were identified. As seen in Table 1, in comparison with references strains of Clostridium butyricum, one SNP was identified in the susceptible strain C. butyricum 1008 Y240D (Tyr240Asp) and two SNPs were identified in the less susceptible strain C. butyricum 1007, Y38D and D146E (Tyr38Asp and Asp146Glu). Results suggest that targeted ibezapolstat drug development towards C. difficile may lead to intermittent activity against beneficial commensals.

### Example 5

Novel Pharmacology and Susceptibility of Ibezapolstat Against C. difficile Isolates With Reduced Susceptibility to C. difficile-Directed Antibiotics

The purpose of this study was to assess the efficacy of ibezapolstat against C. difficile strains that exhibit reduced susceptibility to current CDI antibiotics. Additionally, this study assessed motility inhibition and the effects IBZ treatment exhibited in flagellar gene expression in C. difficile strains with reduced susceptibility to current CDI antibiotics.

### Methods and Materials

The goal of this study was to assess the susceptibility of IBZ against strains with reduced susceptibility to current CDI antibiotics and assess motility inhibition.

### Isolates:

Twelve clinical isolates with reduced susceptibility to metronidazole (MIC range: 0.25-8 ug/mL), vancomycin (MIC range: 1-16 ug/mL), or fidaxomicin (<0.03125-2 ug/mL) were tested.

### Minimum Inhibitory Concentration (MIC) Determinations:

Agar dilution MIC studies were performed against C. difficile strains with reduced susceptibility to metronidazole, vancomycin, and fidaxomicin following guideline of CLSI document M11-A7 for anaerobic bacteria. Cultures of C. difficile were prepared by inoculating BHI supplemented with 0.1% sodium taurocholate with one colony of bacteria. After incubation at 37° C. in an anaerobic chamber for 24-hours, pre-cultures were diluted 1:100 to approximately 10⁶ CFU/mL in fresh BHI including the appropriate concentration of antibiotic. Following CLSI guidelines, antibiotic concentrations were prepared by a 2× dilution series (1 concentration per agar plate). For example, for each of the three antibiotics, i.e. vancomycin, ibezapolstat, or metronidazole, the concentration of antibiotic ranged from about 64 ug/ml to about 0.25 ug/ml, for fidaxomicin the concentration ranged from about 16 ug/ml to about 0.03 ug/ml.

Inhibitory concentrations were determined by eye visualization at 24 hours. As shown in Table 5, MIC values are associated with susceptibility against an antibiotic with reduced susceptibility (difference >8× MIC value) where MIC is expressed in mg/L. The results indicate that IBZ maintains its efficacy against clinical isolates with reduced susceptibility to metronidazole, vancomycin, and fidaxomicin. Further, IBZ MIC50 and MIC90 did not differ between susceptible and reduced susceptible isolates.

TABLE 5
IBZ Retained Activity vs. Resistant Strains

| | Metronidazole | Vancomycin | Fidaxomicin | Ibezapolstat |
|---|---|---|---|---|
| MT 4802 | 0.25 | 1 | 0.006 | 4 |
| MT 5529 | 2 | 1 | 0.5 | 4 |
| SH 1132 | 1 | 4 | 0.5 | 8 |
| MT 5342 | 2 | 8 | 1 | 4 |
| MT 5364 | 2 | 4 | 1 | 4 |
| MT 5426 | 4 | 4 | 0.5 | 8 |
| MT 5515 | 4 | 4 | 2 | 8 |
| MT 4883 | 2 | 8 | 2 | 8 |
| MT 5493 | 2 | 1 | 1 | 4 |
| MT 5536 | 0.25 | 2 | 1 | 8 |
| MT 5382 | 2 | 1 | 1 | 8 |
| MT 5071 | 1 | 4 | 1 | 8 |

Quantitative PCR (qPCR) Analysis

C. difficile motility was assessed using quantitative RT-PCR. Following pre-treatment with sub-MIC concentrations of IBZ adapted from the methodology of Doan et al. (Antibiotics 2022), relevant flagellar gene expression (fliA, flgB, fliC-VIP) in reference C. difficile strain CD 630 was quantified by qPCR. Briefly, bacterial pre-cultures were diluted, then grown in BHI at sub-inhibitory concentrations of IBZ (0.5×MIC) at 37° C. under anaerobic conditions for 4 hours. Transcript levels of fliA, flgB and fliC were determined and compared to control, gluD recombinant protein (Clostridium difficile).

FIG. 3 shows the targeted genes on the x-axis and relative expression of these genes compared to the non-treated control on the y-axis. In addition, to better visualize the data, the relative expression of the control was normalized to the value 1. Accordingly, FIG. 3 shows that the fliA expression was attenuated by about 60%, flgB expression decreased by about 30%, and fliC expression decreased by about 80% when treated with IBZ as compared to the non-treated control. Thus, qPCR analysis revealed between a 2-to-5-fold decrease overall in flagellar gene expression following sub-MIC IBZ exposure.

### Motility Assay:

C. difficile motility was assessed using a phenotypic motility assay. Briefly, cultures of CD 630 were prepared by inoculating BHI medium containing 0.3% agar and sub-inhibitory concentrations of IBZ, and grown anaerobically at 37° C. for 48 hours. Following incubation, the bacterial strains were placed in semi-solid BHI agar for 4 hours and movement was visually observed.

FIG. 4 shows the results of the motility assay. As seen in FIG. 4, the control strain is able to swim without constrains and therefore may be seen moving freely away from the inoculation vertical line. On the other hand, strains exposed to sub-inhibitory (not killing) concentrations of IBZ in the medium may be seen staying close to their inoculation point. Such results indicate that the cultures of CD 630 are less motile due to the presence of IBZ in the medium.

Results showed that IBZ maintained activity against C. difficile strains with reduced susceptibility to other commonly used antibiotics and demonstrated a novel pharmacologic property which is likely due to its unique mechanism of action.

### Example 6

### Metagenomic Evaluation of Ibezapolstat Compared to Other Anti-Clostridioides Difficile Agents

Published studies demonstrate that, in humans, treatment with Ibezapolstat (IBZ) may result in less microbiome disruption than treatment with vancomycin (See e.g., FIG. 5). Despite this, there are no comparative microbiome studies for other anti-C. difficile antibiotics. The purpose of this study was to compare the in vivo changes in gut microbiome in response to treatment with IBZ and other anti-C. difficile antibiotics. Thus, this study set out to compare gut microbiome perturbation from IBZ to three other anti-C. difficile antibiotics including vancomycin (VAN), fidaxomicin (FDX), or metronidazole (MTZ).

### Methods and Materials

Germ-free (GF) mice (six per group) were randomly assigned to IBZ, VAN, FDX, MTZ, or control groups. FIG. 6 shows the basic experimental design of the humanized germ-free mouse study.

### Germ-Free (GF) Humanized Mice

An oral gavage of healthy human-derived fecal slurry was given to GF mice. The humanized mice were housed in a sterile biological safety cabinet (BSC) for one week to allow the donor gut microbiome to establish. Humanized mice were then moved to sterile, micro-isolator cages and pellet chow diet was exchanged for a powder chow diet. The humanized mice were then allowed one week to acclimate to the powder chow diet. In addition, a stool sample was obtained prior to the change in diet (baseline 1). At day 14, once the mice acclimated to the powder chow diet, a second baseline stool sample was obtained (baseline 2) and antibiotic treatment was started. The appropriate antibiotics were added to the powder chow diet for 10 days. At day 16, two days after beginning antibiotic treatment, another stool sample was collected (ABX-1). During the 10-day antibiotic treatment, powder chow dishes were refilled each day with fresh chow containing the randomly assigned antibiotic. Stool samples were again collected at day 24 (ABX-2).

### Microbiome Analysis

To assess antibiotic microbiome effect, 16S rRNA metagenomics was used on stool samples that were collected at day 0 (baseline 1 and baseline 2) and after 2 and 14 days of treatment with antibiotics. Bulk DNA was extracted from stool samples using the DNeasy® PowerSoil® Kit. The V4 region of the bacterial 16S rRNA gene was sequenced using the Illumina MiSeq and CLC Genomics Workbench. Raw sequencing reads were processed and curated using the mothur (v.1.48.0) software package and the mothur MiSeq SOP was followed as outlined in Schloss, P. D., et al., Introducing mothur: open-source, platform-independent, community supported software for describing and comparing microbial communities. Appl Environ Microbiol, 2009. 75(23): p. 7537-41 (see also, Kozich, J. J., et al., Development of a dual-index sequencing strategy and curation pipeline for analyzing amplicon sequence data on the MiSeq Illumina sequencing platform. Appl Environ Microbiol, 2013. 79(17): p. 5112-20.). All data visualization and statistical analysis were performed using R studio (R Core Team (2023). R: A Language and Environment for Statistical Computing, Vienna, Austria.).

### Results

Prior to antibiotic initiation, Shannon's index alpha diversity was similar across treatment groups.

### Microbiome Analysis

FIGS. 7A-E shows box plots with lines connecting groups based on color, where the colors represent the antibiotic to which the mice were exposed during the final 10-days of the experiment (or lack thereof in the cases of the no drug control (ND Control) and baseline samples). Changes in α-diversity of the gut microbiome throughout the experiment are shown in FIGS. 7A and 7B, these figures show the changes in the richness of Operational Taxonomic Units (OTUs) within the gut microbiome over the course of the experiment (FIG. 7A) as well as the changes in the in the inverse-Simpson's Index within the gut microbiome over the course of the experiment (FIG. 7B). For both metrics, the combination of dietary change followed by exposure to different antibiotic significantly impacted the inverse-Simpson's index (ANOVA; p<0.001). As seen in FIGS. 7A and 7B, there was a significant decrease in alpha diversity in all antibiotic groups compared to control (p<0.05).

FIGS. 7C, 7D and 7E demonstrate the changes in β-diversity (diversity between groups) of the gut microbiome throughout the experiment and show distinct clustering in mice given IBZ versus all other antibiotics. FIG. 7C illustrates the changes in beta dispersion (distance-to-centroid) of the gut microbiome throughout the experiment, FIG. 7D illustrates changes in Bray-Curtis Dissimilarity to Baseline 1 (D7) throughout the experiment, and FIG. 7E illustrates changes in Bray-Curtis Dissimilarity to Baseline 2 (D14). In all metrics of β-diversity, a dietary change followed by exposure to an antibiotic significantly increased the Bray-Curtis dissimilarity to Baseline 2 (D14) (ANOVA, p<0.001 for all).

FIG. 7F provides non-metric multidimensional scaling (NMDS) of Bray-Curtis dissimilarity.

The box plots presented in FIGS. 7A-7E include dots that represent one sample taken at the respective time point (labeled at the top of each plot), size indicates the inverse Simpson Index value for that sample, shape indicates which trial the sample was collected from, and color indicates which antibiotic (or lack thereof) each sample was exposed to. When comparing exposure groups (colors) on the two timepoints (ABX-1 and ABX-2) in which there were groups exposed to antibiotics, there were statistical difference between antibiotic exposure groups (PERMANOVA; p<0.001 for both timepoints). Additionally, when comparing Trial 1 and Trial 2, there were statistical differences (PERMANOVA; p<0.001) between the two trials on all timepoints (Baseline 1, Baseline 2, ABX-1, and ABX-2). Table 6 below depicts the changes in metrics of diversity within the gut microbiome observed in the treated humanized mice and indicate comparisons being made between antibiotic treatments. For each antibiotic (ibezapolstat, fidaxomicin, vancomycin, and metronidazole), comparisons were made to Baseline 2 (D14). Comparisons of D7 and D14 to account for shifts in diversity because of the change in diet were made using Baseline 1 (D7) as the reference point. Significant codes were determined based off the following p-value parameters: ˜0=“***”, ˜0.001=“**”, ˜0.01=“*”, ˜0.05=“.” Anything with a p value >0.05 was not considered significant.

TABLE 6
Changes in Metrics of Diversity within the Gut Microbiome

| | | | Inverse | | Bray-Curtis | Bray-Curtis |
|---|---|---|---|---|---|---|
| | | | Simpson's | Beta | Dissim. to | Dissim. to |
| | | OTU Richness | Index | Dispersion | Baseline 1 | Baseline 2 |
| Change in Diet | Average Change in | (−)8.7 ± 1.7 | (−)4.6 ± 0.5 | 0.012 ± 0.01 | 0.58 ± 0.01 | 0.58 ± 0.02 |
| (D 7 v D 14) | Metric ± SD | | | | | |
| | P-value | 2.28E−06 | 1.34E−13 | 0.22 | <2e−16 | <2e−16 |
| | Significant Code | *** | *** | | *** | *** |
| No-Drug Control | Average Change in | 4.3 ± 2.5 | 3.3 ± 0.9 | (−)0.05 ± 0.01 | 0.61 ± 0.02 | 0.32 ± 0.03 |
| | Metric ± SD | | | | | |
| | P-value | 0.08 | 0.002 | 0.002 | <2e−16 | <2e−16 |
| | Significant Code | | *** | *** | *** | *** |
| Ibezapolstat | Average Change in | (−)33.3 ± 2.6 | (−)3.7 ± 0.9 | (−)0.023 ± 0.01 | 0.81 ± 0.02 | 0.68 ± 0.03 |
| | Metric ± SD | | | | | |
| | P-value | <2e−16 | 6.23E−05 | 0.125 | <2e−16 | <2e−16 |
| | Significant Code | *** | *** | | *** | *** |
| Fidaxomicin | Average Change in | (−)22.7 ± 2.5 | (−)3.1 ± 0.9 | (−)0.05 ± 0.01 | 0.62 ± 0.02 | 0.39 ± 0.03 |
| | Metric ± SD | | | | | |
| | P-value | 4.73E−15 | 0.0004 | 0.001 | <2e−16 | <2e−16 |
| | Significant Code | *** | *** | ** | *** | *** |
| Vancomycin | Average Change in | (−)39.1 ± 2.3 | (−)6.7 ± 0.8 | (−)0.01 ± 0.01 | 0.84 ± 0.02 | 0.75 ± 0.03 |
| | Metric ± SD | | | | | |
| | P-value | <2e−16 | 4.23E−13 | 0.51 | <2e−16 | <2e−16 |
| | Significant Code | *** | *** | | *** | *** |
| Metronidazole | Average Change in | (−)38.4 ± 3.1 | (−)7.6 ± 1.1 | (−)0.11 ± 0.02 | 0.92 ± 0.02 | 0.92 ± 0.04 |
| | Metric ± SD | | | | | |
| | P-value | <2e−16 | 1.55E−10 | 2.33E−09 | <2e−16 | <2e−16 |
| | Significant Code | *** | *** | *** | *** | *** |

FIGS. 8A-8D provide stacked bar charts illustrating the mean relative abundance (represented as a percentage) of different bacterial taxonomy levels throughout the experiment. Thus, FIG. 8A represents the phylum level, FIG. 8B represents the class level, FIG. 8C represents the order level, and FIG. 8D represents the family level. In addition, Table 7 below depicts the changes in relative abundance of bacterial phyla within the gut microbiome observed in the treated humanized mice and comparison between the different antibiotic treatments. For each antibiotic (ibezapolstat, fidaxomicin, vancomycin, and metronidazole), comparisons were made to Baseline 2 (D14). Comparisons of D7 and D14 to account for shifts in diversity because of the change in diet were made using Baseline 1 (D7) as the reference point. Significant codes were determined based off the following p-value parameters: ˜0=“***”, ˜0.001=“**”, ˜0.01=“*”, ˜0.05=“.” Anything with a p value >0.05 was not considered significant.

TABLE 7
Changes in Relative Abundance of Bacterial Phyla

| | | | | | | Verrucomicrobia |
|---|---|---|---|---|---|---|
| | | Bacteroidetes | Firmicutes | Proteobacteria | Actinobacteria | (Akkermansia) |
| Change in Diet | Avg. | (−)4.5% ± 2.2% | (−)4.1% ± 3.1% | 2.4% ± 2.2% | (−) 0.08% ± 0.06% | 7.1% ± 1.9% |
| (D 7 v D 14) | Change in | | | | | |
| | Relative | | | | | |
| | Abundance ± | | | | | |
| | SD | | | | | |
| | P-value | 0.0399 | 0.19 | 0.284 | 0.17 | 0.003 |
| | Significant | * | | | | *** |
| | Code | | | | | |
| No-Drug | Avg. | 7.1% ± 2.9% | 4.5% ± 4.1% | (−)0.219% ± 3.02% | 0.04% ± 0.07% | (−)7.5% ± 2.8% |
| Control | Change in | | | | | |
| | Relative | | | | | |
| | Abundance ± | | | | | |
| | SD | | | | | |
| | P-value | 0.014 | 0.28 | 0.942 | 0.608 | 0.009 |
| | Significant | * | | | | ** |
| | Code | | | | | |
| Ibezapolstat | Avg. | 34% ± 2.96% | (−)36.1% ± 4.3% | 9.7% ± 3.12% | 0.02% ± 0.08% | (−)7.1% ± 3% |
| | Change in | | | | | |
| | Relative | | | | | |
| | Abundance ± | | | | | |
| | SD | | | | | |
| | P-value | <2e−16 | 1.17E−13 | 0.003 | 0.74 | 0.02 |
| | Significant | *** | *** | ** | | * |
| | Code | | | | | |
| Fidaxomicin | Avg. | 9.9% ± 2.9% | (−)25% ± 4.13% | 10.2% ± 3% | (−)0.2% ± 0.07% | 3.6% ± 2.8% |
| | Change in | | | | | |
| | Relative | | | | | |
| | Abundance ± | | | | | |
| | SD | | | | | |
| | P-value | 0.0008 | 2.01E−08 | 0.001 | 0.009 | 0.2 |
| | Significant | *** | *** | ** | | |
| | Code | | | | | |
| Vancomycin | Avg. | (−)31.1% ± 2.7% | (−)4.1% ± 3.9% | 37.2% ± 2.8% | (−) 0.19% ± 0.07% | (−)3.5% ± 2.7% |
| | Change in | | | | | |
| | Relative | | | | | |
| | Abundance ± | | | | | |
| | SD | | | | | |
| | P-value | <2e−16 | 0.303 | <2e−15 | 0.009 | 0.2 |
| | Significant | *** | | *** | | |
| | Code | | | | | |
| Metronidazole | Avg. | (−)37.6% ± 3.6% | 36.5% ± 5.1% | 15.1% ± 3.8% | (−) 0.14% ± 0.09% | (−)15.6% ± 3.5% |
| | Change in | | | | | |
| | Relative | | | | | |
| | Abundance ± | | | | | |
| | SD | | | | | |
| | P-value | <2e−16 | 1.31E−10 | 0.001 | 0.13 | 6.60E−05 |
| | Significant | *** | *** | *** | | *** |
| | Code | | | | | |
| Trial 1 vs | Avg. | (−)3.1% ± 1.6% | (−)1.9% ± 2.4% | (−)10.2% ± 1.9% | (−)0.16% ± 0.04% | 15.7% ± 2.4% |
| Trial 2 | Change in | | | | | |
| | Relative | | | | | |
| | Abundance ± | | | | | |
| | SD | | | | | |
| | P-value | 0.07 | 0.427 | 7.11E−06 | 0.001 | 5.44E−07 |
| | Significant | | | *** | ** | *** |
| | Code | | | | | |

FIGS. 9A-9E provide bar charts of OTUs identified by random forest analysis that distinguish specified treatment groups. The dashed lines represent the significance cutoff based on the median importance level given 1 standard deviation in both directions and OTUs shown (x-axis) are classified to the family level. In addition, comparisons shown in legend and enriched (elevated) OTUs are colored for each panel. FIGS. 9A-D depict comparisons of each antibiotic exposed mice to the no drug control group (ND Control) and FIG. 9E compares mice exposed to ibezapolstat to mice exposed to fidaxomicin.

At the phylum level, a significant proportional increase of Bacteroidetes was observed in the IBZ group, whereas increased Firmicutes were observed in the FDX group. Further, the relative abundances of Proteobacteria and Verrucomicrobiota increased in VAN and MTZ groups.

### Conclusions

These studies show that changes in food from pellet chow to powder chow decreased α-diversity, causing a slight decrease in Bacteroidetes abundance and an increase in Verrucomicrobia (Akkermansia). Although all antibiotics perturbed the gut microbiome, changes appear to be drug-dependent. In general, ibezapolstat and fidaxomicin caused proportional increases in Bacteroidetes while vancomycin and metronidazole caused proportional increases in Proteobacteria.

Moreover, IBZ and fidaxomicin given to human microbiome GF mice both resulted in favorable yet distinctive changes in the gut microbiome compared to the well-known gut microbiota disrupting agents, vancomycin and metronidazole. These results support the continued clinical development of IBZ for the treatment of CDI.

### Example 7

### Ibezapolstat Modulates Clostridioides Difficile Virulence Factors In Vitro

In phase I and phase Ila clinical trials performed to date, use of ibezapolstat has shown a favourable effect on the gut microbiome that may predict an anti-recurrence pharmacologic property. Thus, the goal of this study was to assess the impact of IBZ on C. difficile virulence factors. To facilitate this goal, the study investigated the in vitro impact of sub-inhibitory concentrations of IBZ on cell morphology, motility and toxin production in C. difficile. Based on its potentially novel mechanism of action that targets the DNA pol IIIC enzyme, IBZ may demonstrate unique pharmacologic properties beyond bacterial killing.

### Methods and Materials

C. difficile reference strains CD 630 and R20291 were treated with sub-inhibitory to minimum inhibitory concentrations of ibezapolstat. Toxin A and B concentrations were measured by ELISA (tgcBiomics). The expression of flagellar genes fliA, flgB and fliC in CD 630 was assessed after 4 hrs of treatment with IBZ using RT qPCR. Morphology changes induced by treatment with IBZ were evaluated by bright field microscopy at 10-40× magnification.

### Morphology Assessment

Cultures were prepared by inoculating Brain Heart Infusion broth (BHI) supplemented with 0.1% sodium taurocholate with one colony of C. difficile. Cultures were then incubated in an anaerobic chamber at 37° C. in for 24 hrs. Pre-cultures were diluted to approximately 10⁶CFU/mL (1:100) in fresh BHI comprising 0.25×MIC (0.5 ug/mL) of the appropriate antibiotic. Following incubation for 24 hrs, EVOS imaging system (Thermofisher) was used to take bright field pictures at 40× magnification.

### Motility Assay

C. difficile motility was assessed using a phenotypic motility assay. Cultures of bacterial strains were prepared by inoculating BHI medium containing 0.3% agar and sub-inhibitory concentrations of IBZ, and grown anaerobically at 37° C. for 48 hours. Following incubation, the bacterial strains were placed in semi-solid BHI agar for 76 hours and movement was visually observed.

### Expression of Flagellar Genes

Bacterial pre-cultures were diluted in BHI and grown at 37° C. under anaerobic conditions for 4 hours with sub-inhibitory concentrations of antibiotic. To assess flagellar gene expression, transcripts levels of fliA, flgB and fliC were measured by qRT-PCR (Doan et al. 2022).

### Toxin Production

Bacterial precultures were diluted then grown in BHI at sub-inhibitory concentrations of antibiotic for 24 h. Toxin production was assessed by ELISA (tgcBiomics) according to the manufacturer instructions.

### Results

In general, IBZ decreased toxin A and B concentrations in a dose-dependent manner. After normalizing toxin production to controls, a reduction of toxin levels of 55% (CD 630) to 60% (R20291) was observed. Also observed, an up to 50% reduction in motility and flagellar genes following a 4-hour treatment with 0.25×MIC IBZ. And lastly, both CD 630 and R20291 strains exhibited an elongated cell phenotype at sub-inhibitory to MIC of IBZ.

### Morphology Assessment

FIG. 10 shows the effect of Ibezapolstat on CD 630 morphology. As shown in FIG. 10, IBZ displays a dose-dependent effect on C. difficile cell length that affects its cell division pathway after 24 hours of treatment. This phenotype has been previously observed with other antibiotics targeting DNA.

### Motility Assay

FIG. 11 shows the effects of Ibezapolstat on CD 630 motility. As seen in FIG. 11, CD 630 treated with sub-inhibitory concentrations of ibezapolstat in semi-solid BHI agar for at least 48 hours (up to 76 hours) resulted in attenuated motility.

### Expression of Flagellar Genes

FIG. 12 shows the effects of Ibezapolstat on flagellar gene expression. Following a 4-hour treatment with sub-inhibitory concentration of IBZ (0.5×MIC), CD 630 demonstrated a 2-to-5-fold decrease of flagellar genes as compared to the gluD control (FIG. 12).

### Toxin Production

FIG. 13 shows the effects of Ibezapolstat exerts on modulating C. difficile toxin production. As FIG. 13 demonstrates, IBZ decreases total toxin A and B production in strain CD 630 in a dose-dependent manner following 24 hours of treatment.

### Conclusion

As shown in FIGS. 10-13, Ibezapolstat treatment exhibits in vitro effects on virulence determinants of C. difficile. At sub-inhibitory levels, Ibezapolstat impacts several aspects of C. difficile virulence. For example, IBZ affects bacterial fitness through modulation of morphology leading to cell division impairment. IBZ also reduces C. difficile motility as visualized in the motility assay seen in FIG. 11. And further, IBZ attenuated the relative expression of flagellar genes fliA, flgB and fliC and reduced C. difficile production of both toxin A and toxin B. These preliminary results elucidate IBZs mechanism of action and support it continued clinical development.

### Example 8

C. difficile In Vitro Biofilm Studies of Ibezapolstat and Comparator Antibiotics

The high rate of Clostridioides difficile infection (CDI) recurrence is hypothesized to be partly attributed to biofilm formation. Although IBZ has been shown to be a novel pol IIIC DNA polymerase inhibitor antibiotic with low rates of CDI recurrence in Phase II clinical trials, its in vitro effects on C. difficile biofilms are unknown. The objective of this study, therefore, was to compare IBZs C. difficile anti-biofilm activity with comparator antibiotics.

### Methods and Materials

To observe a compare the effects of IBZ and comparable antibiotics on C. difficile biofilms, antimicrobial activity and biofilm biomass studies were used. In these studies, IBZ was compared to antimicrobials vancomycin (VAN), fidaxomicin (FDX), and metronidazole (MTZ). A basic flow chart outlining the experimental procedure used in these studies may be seen in FIG. 14.

### Biofilm Formation

Briefly, C. difficile laboratory strain R20291 was grown in Brain heart infusion-supplemented (BHIS) medium. To start the biofilm culture 100 μL of C. diff. culture (OD600 nm=0.01) was added to each well of a 96-well plate. Biofilm formation was established via anaerobic incubation of the cultures at 37° C. for 24 hrs. Early biofilm formation was established at 4 hrs, whereas late biofilm formation was established at 24 hrs, 48 hrs, and 72 hrs.

### Antimicrobial Activity

Following biofilm formation, the BHIS medium was removed and replaced with fresh media comprising no antibiotic (control), ibezapolstat (IBZ), Fidaxomicin (FDX), or metronidazole (MTZ). The cultures were then anaerobically incubated for 24 hrs or subjected to time course tests.

### Biofilm Biomass

Biofilm biomass was measured using Crystal Violet staining. Cultures were stained with 0.2% Crystal Violet for 30 minutes. Data was measured and recorded using Cytation 3 calibrated to read at A570. Data was analyzed by comparing percent growth of antibiotic treated cultures with controls.

### Results

FIG. 15 shows early (24 hour) and late (>72 hour) biofilm formation for biofilms embedded with either R20291 or CD 630 C. diff strains. FIGS. 17A and 17B demonstrate that IBZ and vancomycin (VAN) inhibit C. diff biofilm formation and reduce C. diff biofilm biomass at multiple time points. Compared to control, IBZ and VAN reduced C. diff growth (CFU/mL) and biomass at sub-MIC (0.4× MIC) and in early biofilms supra-MIC (40× MIC) of VAN reduced growth whereas IBZ eradicated growth after 48 hours of treatment (See FIGS. 17A and 17B).

### Antimicrobial Activity

FIG. 16 shows 24-hour biofilms embedded with either R20291 or CD 630 C. diff strains. As seen in FIG. 16, minimum bactericidal concentrations were similar to vegetative, non-biofilm growth for all four antibiotics although an Eagle effect was observed for MTZ at higher concentrations.

### Biofilm Biomass

As mentioned above, FIGS. 17A and 17B show the effects of IBZ and VAN on early biofilms (i.e. biofilms formed in 4 hours). As seen in FIG. 17A both IBZ and VAN, at MIC and sub-MIC levels, reduced biofilm growth within 48 hours as measured in CFU/mL. In addition, FIG. 18 shows that late biofilms (i.e. biofilms formed in >24 hours) embedded with R20291 or CD 630 demonstrate biphasic growth with reduced biomass at 48 hour and 24 hours with regrowth observed (likely due to biofilm detachment) at 72 hours. As shown in FIG. 18, although differences were noted, all antibiotics in general reduced biofilm biomass compared to control regardless of biofilm growth time with highest reductions observed at later biofilm growth periods (48 h and 72 h). In addition, Table 8 provides MIC and MBIC for the tested antibiotics in μg/mL as applicable to C. diff. strain R20291.

TABLE 8

| Compound | MIC (μg/mL) | MBIC (μg/mL) |
|---|---|---|
| Vancomycin | 1 | 1 |
| Ibezapolstat | 4 | 4 |
| Fidaxomicin | 0.06 | 0.125 |
| Metronidazole | 0.5 | 0.5 |

### Conclusion

These studies demonstrate that IBZ may be as effective as comparable antibiotics in reducing both quantity and biofilm biomass in biofilm-embedded C. difficile. These results, together with clinical trial success to date, warrant the continued development of IBZ.

### OTHER EMBODIMENTS

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention. The contents of all references, issued patents, and published patent applications cited through this application are hereby incorporated by reference. The appropriate component, process and methods of those patents, applications and other documents may be selected for the invention and embodiments thereof.

### REFERENCES

1. Mullish B H, Quraishi M N, Segal J P, et al., The gut microbiome: what every gastroenterologist needs to knowFrontline Gastroenterology 2021; 12:118-127.
2. Kachrimanidou M. Tsintarakis E. Insights into the Role of Human Gut Microbiota in Clostridioides difficile Infection. Microorganisms. 2020; 8(2):200. https://doi.org/10.3390/microorganisms8020200.
3. Mueller N T, et al., The infant microbiome development: mom matters. Trends Mol Med. 2015 February; 21(2):109-17. doi: 10.1016/j.molmed.2014.12.002. Epub 2014 Dec. 11. PMID: 25578246; PMCID: PMC4464665.
4. Jangi, S. and Lamont, T., (2010) Asymptomatic Colonization by Clostridium difficile in Infants: Implications for Disease in Later Life, Journal of Pediatric Gastroenterology and Nutrition. 51(1):2-7.
5. Huemer, M. et al., Antibiotic resistance and persistence-Implications for human health and treatment perspectives, EMBO Rep. 2020 Dec. 3; 21(12): e51034.
6. Tanwar J. Das S, Fatima Z, Hameed S (2014) Multidrug resistance: an emerging crisis. Interdiscip Perspect Infect Dis 2014:541340.
7. Spellberg B, Bartlett J G, Gilbert D N (2013) The future of antibiotics and resistance. N Engl J Med 368:299-302.
8. Rice L B (2010) Progress and challenges in implementing the research on ESKAPE pathogens. Infect Control Hosp Epidemiol 31 (Suppl 1): S7-10.
9. Magill, et al., “Changes in Prevalence of Health Care-Associated Infections in U.S. Hospitals” The New England Journal of Medicine 2018, 379, 1732-1744.
10. Li, W., et al., (2022), Evaluation of culturable ‘last-resort’ antibiotic resistant pathogens in hospital wastewater and implications on the risks of nosocomial antimicrobial resistance prevalence. Journal of hazardous materials, 438, 129477. https:/doi.org/10.1016/j.jhazmat.2022.129477
11. Davis M L, et al. Multicentre derivation and validation of a simple predictive index for healthcare-associated C. difficile infection. (Clin Microbiol Infect 2018; 24:1190-4).
12. Chambers H F, Deleo F R (2009) Waves of resistance: Staphylococcus aureus in the antibiotic era. Nat Rev Microbiol 7:629-641.
13. Isaac S, et al., Short- and long-term effects of oral vancomycin on the human intestinal microbiota. J Antimicrob Chemother 2017; 72:128-36.
14. Peng Z, et al., Update on antimicrobial resistance in C. difficile: resistance mechanisms and antimicrobial susceptibility testing. (J Clin Microbiol 2017; 55:1998-2008).
15. Garey K W, et al. A randomized, double-blind, placebo-controlled, single and multiple ascending dose Phase 1 study to determine the safety, pharmacokinetics and food and faccal microbiome effects of ibezapolstat administered orally to healthy subjects. J Antimicrob Chemother 2020; 75 (12): 3635-3643. DOI: 10.1093/jac/dkaa364.
16. Gonzales-Luna A J, Carlson T J. Dotson K M, et al. PCR ribotypes of Clostridioides difficile across Texas from 2011 to 2018 including emergence of ribotype 255. Emerg Microbes Infect 2020; 9(1):341-7.
17. Kornberg, et al., “DNA replication” 1992, W. H. Freeman & Co., New York, N.Y.
18. Tarantino, et al., “Inhibitors of DNA polymerase III as novel antimicrobial agents against Gram-positive cubacteria” Antimicr. Agents Chemother. 1999, 43, 1982-1987.
19. Begum K, Basseres E, Miranda J, et al. In Vitro Activity of Omadacycline, a New Tetracycline Analog, and Comparators against Clostridioides difficile. Antimicrob Agents Chemother 2020; 64 (8).

### OTHER REFERENCES

Wright, et al., “Inhibition of Bacillus subtilis DNA Polymerase III by Arylhydrazinopyrimidine” Biochimica et Biophysica Acta 1976, 432, 37-48
Wright, et al., “DNA polymerase III: A new target for antibiotic development”, Current Op. Anti-Infective Investigat. Drugs 1999, 1, 45-48.
Tarantino, et al., “6-Anilinouracil-Based Inhibitors of Bacillus subtilis DNA Polymerase III: Antipolymerase and Antimicrobial Structure-Activity Relationships Based on Substitution at Uracil N3” J. Med. Chem. 1999, 42, 2035-2040.
Daly, et al., “In vitro antimicrobial activity of novel anilinouracils which selectively inhibit DNA polymerase III of Gram-positive bacteria” Antimicr. Agents Chemother. 2000, 44, 2217-2221.
Ali, et al., “Design and Synthesis of Novel Antibacterial Agents with Inhibitory Activity against DNA Polymerase III” Bioorg. Med. Chem. Lett. 2001, 11, 2185-2188.
Muto, et al., “SHEA Guideline for Preventing Nosocomial Transmission of Multidrug-Resistant Strains of Staphylococcus aureus and Enterococcus” Infection Control and Hospital Epidemiology 2003, 362-386.
Ali, et al., “Novel Pyrazolo[3,4-d]pyrimidine-Based Inhibitors of Staphylococcus aureus DNA Polymerase III: Design, Synthesis, and Biological Evaluation” J. Med. Chem. 2003, 46, 1824-1830.
Zhi, et al., “Synthesis and Antibacterial Activity of 3-Substituted-6-(3-ethyl-4-methylanilino) uracils” J. Med. Chem. 2005, 48, 7063-7074.
Wright. et al., “Active site directed inhibitors of replication-specific bacterial DNA polymerases” Bioorg Med Chem Lett. 2005, 15, 729-732.
Kuhl, et al., “Biological Characterization of Novel Inhibitors of the Gram-Positive DNA Polymerase IIIC Enzyme” Antimicr. Agents Chemother. 2005, 49, 987-995.
Wright, et al., “Purine and Isosteric Antibacterial Compounds” U.S. Pat. No. 6,926,763 (2005)
Zhi, et al., “Hybrid Antibacterials. DNA polymerase topoisomerase inhibitors.” J. Med. Chem. 2006, 49, 1455-1465.
Evans, et al., “Structure of Po/C reveals unique DNA binding and fidelity determinants” Proc Natl Acad Sci USA 2008, 105, 20695-700.
Cer, et al., “IC₅₀-to-Ki: a web-based tool for converting IC₅₀ to Ki values for inhibitors of enzyme activity and ligand binding” Nucleic Acids Research, 2009, Vol. 37, Web Server issue W441-W445 doi: 10.1093/nar/gkp253
Hookman. et al., “Clostridium difficile associated infection, diarrhea and colitis,” World J. Gastroenterol 2009, 15, 1554-1580.
Torti, et al., “Clostridium difficile DNA polymerase IIIC: Basis for activity of Anti-Bacterial Compounds” Current Enzyme Inhibition 2011, 7, 147-153.
Xu, et al., “7-Alkyl-N2-substituted-3-deazaguanines. Synthesis, DNA polymerase III inhibition and antibacterial activity” Bioorg Med Chem Lett. 2011, 21, 4197-202.
Dvoskin, et al., “A novel agent effective against infection with Clostridium difficile” Antimicr. Agents Chemother. 2012, 56, 1624-1626.
Guile, et al., “Antibacterial sulfone and sulfoxide substituted heterocyclic urea compounds” U.S. Pat. No. 8,293,919 (2012),
Guile, et al., “Antibacterial heterocyclic ureas” U.S. Pat. No. 8,716,320 (2014).
Wright, et al., “Selective Antibacterials for Clostridium difficile Infections” U.S. Pat. No. 8,796,292 (2014).
Schloss, P. D., et al., Introducing mothur: open-source, platform-independent, community supported software for describing and comparing microbial communities. Appl Environ Microbiol, 2009. 75(23): p. 7537-41.
Kozich, J. J., et al., Development of a dual-index sequencing strategy and curation pipeline for analyzing amplicon sequence data on the MiSeq Illumina sequencing platform. Appl Environ Microbiol, 2013. 79(17): p. 5112-20.).
R Core Team (2023). R: A Language and Environment for Statistical Computing, Vienna, Austria.).

What is claimed is:

1. A method of promoting gut microbiome health in a subject, comprising:
administering an effective amount of a Low G+C Directed Nucleoside (LDN) Analog to a subject suffering from dysbiosis of the gut;
wherein the LDN analog simultaneously reduces harmful Gram-positive bacteria which genomes comprise low guanine and cytosine (low G+C) content in the gut microbiome while maintaining and/or increasing beneficial microorganisms in the gut microbiome.

2. The method of claim 1, wherein the LDN analog is a small-molecule inhibitor of DNA pol IIIC enzyme.

3. The method of claim 2, wherein LDN analog is directed to the DNA pol IIIC of low G+C class bacteria whose genomes contain <50% guanine (G)+cytosine (C).

4. The method of claim 1, wherein the LDN analog selectively targets physiologically harmful species belonging to Streptococcus, Enterococcus, Staphylococcus, Bacillus, Clostridioides, Pneumococcus, Listeria, Mycoplasma, and/or Lactobacillus.

5. The method of claim 1, wherein the beneficial microorganisms comprise Firmicutes and include Lachnospiraceae and Lactobacilluseae.

6. The method of claim 5, wherein administering the effective amount of the LDN analog reduces the growth or prevents regrowth of low G+C content Gram-positive pathobionts in the gut microbiome within 30 days.

7. A method of achieving and/or maintaining healthy proportions of gut microflora in an intestinal milieu of a subject, comprising:
administering an effective amount of a compound directed against DNA pol IIIC enzyme in low G+C content Gram-positive pathobionts to the subject;
reducing or eliminating physiologically harmful pathogenic microorganisms belonging to phylum Bacillota; and
increasing and/or maintaining physiologically beneficial microorganisms in the intestinal milieu.

8. The method of claim 7, wherein the compound is Low G+C Directed Nucleoside (LDN) Analog.

9. The method of claim 8, wherein the physiologically harmful pathogenic microorganisms include species belonging to Firmicutes and/or Bacillales.

10. The method of claim 8, wherein the physiologically beneficial microorganisms in the intestinal milieu comprise members of phylum Actinomycetota and include Lachnospiraceae and/or Lactobacilluseae.

11. The method of claim 8, wherein administering the LDN analog is prophylactic, the subject is healthy, and the LDN analog restores or maintains a mutualistic relationship between the subject and microorganisms in the intestinal milieu.

12. The method of claim 7, wherein the proportions of phyla of bacteria in the subject's gut microbiome are adjusted to a healthier balance compared to the subject's gut microbiome prior to the administration of the LDN analog.

13. The method of claim 8, wherein the physiologically beneficial microorganisms in the intestinal milieu comprise anaerobic Gram-positive bacterium belonging to genus Clostridium and include C. coccoides.

14. The method of claim 12, wherein the subject is suffering from over-growth of Clostridioides difficile in the gut.

15. The method of claim 11, wherein the administering the LDN analog is continued until a majority proportion of bacterial species is from the Actinobacteria, Firmicute or Bacteroidetes phylum with a minority from the Proteobacteria phylum.

16. A composition for promoting gut microbiome health comprising a Low G+C Directed Nucleoside (LDN) Analog, wherein the LDN analog inhibits DNA pol IIIC enzyme in physiologically deleterious pathobionts, thereby reducing harmful Gram-positive bacteria while allowing beneficial microorganisms to grow in the gut microbiome.

17. The composition of claim 16, wherein the LDN analog is a prophylactic treatment and promotes persistence and/or regrowth of healthy microbiota.

18. The composition of claim 16, wherein the subject receives at least 450 mg of the LDN analog at least once daily.

19. The composition of claim 16, wherein the LDN analog selectively reduces the growth of pathogenic members of Streptococcus, Enterococcus, Staphylococcus, Bacillus, Clostridioides, Pneumococcus, Listeria, Mycoplasma, and/or Lactobacillus.

20. A composition for reducing physiologically harmful gram-positive organisms in a gut microbiome, the composition comprising a Low G+C Directed Nucleoside (LDN) Analog, wherein the LDN analog inhibits motility of the gram-positive organisms.

21. The composition of claim 20, wherein the LDN analog inhibits motility of the gram-positive organisms by reducing flagellar gene expression.
